SEQUENCE LISTING (12 sequences)

SEQ ID NO: 1 - DNA, 273 bp, Homo sapiens
catccggacaaagcctgcgcgcgccccgccccgccattggccgtaccgccccgcgccgccgccccatctcgcccctcgccgccgggtccggcgcgttaaagccaataggaaccgccgccgttgttcccgtcacggccggggcagccaattgtggcggcgctcggcggctcgtggctctttcgcggcaaaaaggatttggcgcgtaaaagtggccgggactttgcaggcagcggcggccgggggcggagcgggatcgagccctcgatgatatca

SEQ ID NO: 2 - DNA, 397 bp, Homo sapiens
ccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcg

SEQ ID NO: 3 - DNA, 245 bp, Homo sapiens
ccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcg

SEQ ID NO: 4 - DNA, 2055 bp, Artificial Sequence
  1 to 2055 of Ar20-1007 including ITR, packaging signal, poly A, E 2F-1 promoter, E1a gene and a portion of the E1b gene
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcaggaaaactgaataagaggaagtgaaatctgaataattttgtgttactcatagcgcgtaatatttgtctagggccgcgggggatctctgcaggaatttgatatcaagcttatcgataccgtcgaaacttgtttattgcagcttataatggttacaaataaagcaatagcatcacaaatttcacaaataaagcatttttttcactgcattctagttgtggtttgtccaaactcatcaatgtatcttatcatgtctggatccgctagcggcgcgccgtttcatccggacaaagcctgcgcgcgccccgccccgccattggccgtaccgccccgcgccgccgccccatctcgcccctcgccgccgggtccggcgcgttaaagccaataggaaccgccgccgttgttcccgtcacggccggggcagccaattgtggcggcgctcggcggctcgtggctctttcgcggcaaaaaggatttggcgcgtaaaagtggccgggactttgcaggcagcggcggccgggggcggagcgggatcgagccctcgatgatatcagaaacgatatcaccggtcgactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaa

SEQ ID NO: 5 - DNA, 1172 bp, artificial sequence
  28781 to 29952 of Ar20-1007 including the E3-6.7 gene, and the human GM-CSF cDNA
acaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagataaccatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagactatcacctttgaaagtttcaaagagaacctgaaggactttctgcttgtcatcccctttgactgctgggagccagtccaggagtaatttactaagttacaaagctaatgtcaccactaactgctttacccgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcatt

SEQ ID NO: 6 - protein, 144 aa, Homo sapiens [sequence omitted]

SEQ ID NO: 7 - DNA, 830 bp, artificial sequence
  28827 to 29656 of Ar20-1004 which includes a sequence encoding a mouse GM-CSF
cagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagataaccatggcccacgagagaaaggctaaggtcctgaggaggatgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacattgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccagtccaaaaataatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggat

SEQ ID NO: 8 - protein, 153 aa, mouse
MAHERKAKVLRRMWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQK

SEQ ID NO: 9 - DNA, 2038 bp, artificial sequence
  1 to 2038 of Ar20-1006 including an ITR, packaging signal, poly A, hTERT promoter, E1a gene and a portion of the E1b gene.
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcaggaaaactgaataagaggaagtgaaatctgaataattttgtgttactcatagcgcgtaatatttgtctagggccgcgggggatctctgcaggaatttgatatcaagcttatcgataccgtcgaaacttgtttattgcagcttataatggttacaaataaagcaatagcatcacaaatttcacaaataaagcatttttttcactgcattctagttgtggtttgtccaaactcatcaatgtatcttatcatgtctggatccgctagccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcgaattaaacgatatcaccggtcgactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaa

SEQ ID NO: 10 - DNA, 900 bp, artificial sequence
  28772 to 29671 of Ar20-1006 which includes the E3-6.7 gene, human GM-CSF cDNA and a portion of the ADP gene.
taggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagataaccatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagactatcacctttgaaagtttcaaagagaacctgaaggactttctgcttgtcatcccctttgactgctgggagccagtccaggagtaatttactaagttacaaagctaatgtcaccactaactgctttacccgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttc

SEQ ID NO: 11 - DNA, 2041 bp, artificial sequence
  1 to 2041 of Ar20-1010, including an ITR, packaging signal, poly A, hTERT promoter, E1a gene and a portion of the E1b gene
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcgggggatctctgcaggaatttgatatcaagcttatcgataccgtcgaaacttgtttattgcagcttataatggttacaaataaagcaatagcatcacaaatttcacaaataaagcatttttttcactgcattctagttgtggtttgtccaaactcatcaatgtatcttatcatgtctggatccgctagccccacgtggcggagggactggggacccgggcaccggtcctgccccttcaccttccagctccgcctcctccgcgcggaccccgccccgtcccgacccctcccgggtccccggcccagccccttccgggccatcccagcccctccccttcctttccgcggccccgccctctcctcgcggcgcgagtttcaggcagcgctgcgtcctgctgcgcacgtgggaagccctggccccggccacccccgcgaattaaacgatatcaccggtcgactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacag

SEQ ID NO: 12 - DNA, 795 bp, artificial sequence
  28781 to 29575 of Ar20-1010 containing the E3-6.7 gene and the mouse GM-CSF cDNA
ccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagataaccatggcccacgagagaaaggctaaggtcctgaggaggatgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacattgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccagtccaaaaataatttactaagttacaaagctaatgtcaccactaactgctttactcgctgcttgcaaaacaaattcaaaaagttagcattataattagaataggatttaaaccccccggtcattt

(19) United States
(12) Patent Application Publication
(10) Pub. No.: US 2008/0118470 A1
(43) Pub. Date: May 22, 2008

(54) Oncolytic adenoviral vectors encoding GM-CSF

(75) Inventor: David Leonard Ennist, Bethesda, MD (US)

Correspondence Address: ROPES & GRAY LLP, PATENT DOCKETING 39/361, 1211 AVENUE OF THE AMERICAS, NEW YORK, NY, 10036-8704, US

(73) Assignee: Cell Genesys, Inc., South San Francisco, CA (US)

(21) Appl. No.: 11/982,995

(22) Filed: Nov. 5, 2007

Related U.S. Application Data
(63) Continuation of application No. 10/925,205, filed on Aug. 23, 2004, now abandoned.
(60) Provisional application No. 60/499,312, filed on Aug. 28, 2003.

(51) Int. Cl. A61K 48/00; C12N 15/861

(52) U.S. Cl. 424/93.2; 435/456; 977/804

(57) ABSTRACT

Selectively replicating oncolytic adenoviral vectors comprising an adenoviral packaging signal, a termination signal sequence, an E2F responsive promoter operably linked to an adenoviral coding region, a heterologous coding sequence encoding GM-CSF and a right ITR are provided. The oncolytic adenoviral vectors are useful for expressing GM-CSF in transduced cells and in methods for selectively killing neoplastic cells.

### FIELD OF THE INVENTION

The present invention generally relates to substances and methods useful for the treatment of neoplastic disease. More specifically, it relates to an oncolytic vector encoding for GM-CSF. The oncolytic adenoviral vectors are useful for expressing GM-CSF from cells and include methods of gene therapy. The oncolytic adenoviral vectors are also useful in methods of screening for compounds that modulate the expression of cancer selective genes that inhibit or enhance the activity of GM-CSF.

### BACKGROUND OF THE INVENTION

The publications and other materials including all patents, patent applications, publications (including published patent applications), and database accession numbers referred to in this specification are used herein to illuminate the background of the invention, and in particular, cases to provide additional details respecting the practice, are incorporated herein by reference to the same extent as if each were specifically and individually indicated to be incorporated by reference in its entirety.

Adenoviruses that replicate selectively in tumor cells are being developed as anticancer agents (“oncolytic adenoviruses”). Such oncolytic adenoviruses amplify the input virus dose due to viral replication in the tumor, leading to spread of the virus in the tumor mass. In situ replication of adenoviruses leads to cell lysis. This in situ replication may allow relatively low, non-toxic doses to be highly effective in the selective elimination of tumor cells.

An approach to achieving selectivity is to use tumor-selective promoters to control the expression of viral genes required for replication. (See, e.g., WO 96/17053, WO 99/25860, WO 02/067861, WO 02/068627, and U.S. Pat. Nos. 5,698,443, 5,871,726, 5,998,205, and 6,432,700, all of which are incorporated herein by reference). Thus, in this approach the adenoviruses will selectively replicate and lyse tumor cells if the gene/coding region that is essential for replication is under the control of a promoter or other transcriptional regulatory element that is tumor-selective.

### SUMMARY OF THE INVENTION

In one aspect, the present invention provides tumor-selective oncolytic adenoviruses armed with the capability of expressing either human or mouse granulocyte-macrophage colony stimulating factor (GM-CSF), exemplified herein by Ar20-1004, Ar20-1006, Ar20-1007 and Ar20-1010. Due to the presence of the tumor-selective E2F-1 promoter, Ar20-1007 and Ar20-1004 will selectively replicate in and selectively kill tumor cells with Rb-pathway defects. Due to the presence of a tumor-selective human telomerase reverse transcriptase (hTERT) promoter, Ar20-1006 and Ar20-1010 will replicate in and selectively kill tumor cells that have up-regulated expression of telomerase.

Ar20-1004, Ar20-1006, Ar20-1007 and Ar20-1010 can selectively kill tumor cells while producing GM-CSF, which is expected to stimulate immune responses against distant uninfected metastases (referred to herein as a bystander effect). These viral vectors contain the majority of the adenovirus E3 region genes and express GM-CSF under the control of the E3 promoter. In a related aspect, the invention provides selective expression of replication competent adenoviral vectors such as those described herein. The viral vectors of the invention may express toxic viral proteins, cause replication and cytolysis of target cells and enhance sensitivity to chemotherapy, cytokines and cytotoxic T lymphocytes (CTL).

In another aspect, the present invention provides a recombinant viral vector comprising in sequential order an adenoviral nucleic acid backbone comprising: a left ITR, an adenoviral packaging signal, a termination signal sequence, an E2F responsive promoter operatively linked an E1a coding region, a sequence encoding a therapeutic gene such as a cytokine, e.g., GM-CSF, and a right ITR.

In another aspect, the present invention provides a recombinant viral vector comprising in sequential order an adenoviral nucleic acid backbone comprising: a left ITR, an adenoviral packaging signal, a termination signal sequence, a telomerase reverse transcriptase (TERT) promoter operatively linked an E1a coding region, a sequence encoding a therapeutic gene such as a cytokine, e.g., GM-CSF, and a right ITR.

In one embodiment, the recombinant viral vector of the present invention is selected from Ar20-1004, Ar20-1006, Ar20-1007 and Ar20-1010.

In another embodiment, the termination signal sequence is an SV40 early polyadenylation signal sequence.

In yet another embodiment of the invention, the E2F promoter is a human E2F promoter. In another embodiment of the invention, the E2F promoter comprises a nucleotide sequence selected from the group consisting of: (a) the sequence shown in SEQ ID NO: 1; (b) a fragment of the sequence shown in SEQ ID NO: 1, wherein the fragment has tumor selective promoter activity; (c) a nucleotide sequence having at least 90% identity over its entire length to the sequence shown in SEQ ID NO: 1, wherein the nucleotide sequence has tumor selective promoter activity; and (d) a nucleotide sequence having a full-length complement that hybridizes under stringent conditions to the sequence shown in SEQ ID NO: 1, wherein the nucleotide sequence has tumor selective promoter activity. In another embodiment of a recombinant viral vector of the invention, the E2F promoter consists essentially of SEQ ID NO:1.

In still another embodiment of the invention, the TERT promoter is a human TERT promoter. In one embodiment of the invention, the TERT promoter comprises a nucleotide sequence selected from the group consisting of: (a) the sequence shown in SEQ ID NO:2; (b) a fragment of the sequence shown in SEQ ID NO:2, wherein the fragment has tumor selective promoter activity; (c) the sequence shown in SEQ ID NO:3; (d) a fragment of the sequence shown in SEQ ID NO: 3, wherein the fragment has tumor selective promoter activity; (e) a nucleotide sequence having at least 90% identity over its entire length to the sequence shown in SEQ ID NO:2 and/or SEQ ID NO: 3, wherein the nucleotide sequence has tumor selective promoter activity; and (f) a nucleotide sequence having a full-length complement that hybridizes under stringent conditions to the sequence shown in SEQ ID NO:2 and/or SEQ ID NO: 3, wherein the nucleotide sequence has tumor selective promoter activity. In another embodiment of a recombinant viral vector of the invention, the TERT promoter consists essentially of SEQ ID NO:2 or SEQ ID NO: 3.

In one further embodiment of the invention, the adenoviral nucleic acid backbone, the left ITR, the adenoviral packaging signal, the E1a coding region and the right ITR are derived from adenovirus serotype 5 (Ad5). In another embodiment of the invention, the adenoviral nucleic acid backbone, the left ITR, the adenoviral packaging signal, the E1a coding region and the right ITR are derived from adenovirus serotype 35 (Ad35). In yet another embodiment of the invention, a portion of the adenoviral nucleic acid backbone, the left ITR, the adenoviral packaging signal, the E1a coding region and the right ITR are derived from one adenovirus serotype, e.g. Ad5 and another portion is derived from Ad35.

In one embodiment, the heterologous coding sequence encoding GM-CSF is inserted in the E3 region of the adenoviral nucleic acid backbone. For example, the heterologous coding sequence may be inserted in place of the 19 kD or 14.7 kD E3 gene.

In one embodiment, the recombinant viral vector, comprises a mutation or deletion in the E1b gene and/or E1b coding sequence. In one embodiment the mutation or deletion results in the loss of the active 19 kD protein expressed by the wild-type E1b gene.

In one embodiment, the recombinant viral vector of the present invention, is capable of selectively replicating in and lysing Rb-pathway defective cells.

In one embodiment, a recombinant viral vector of the invention selectively replicates in tumor cells.

In still another aspect, the present invention provides a method of selectively killing a neoplastic cell, comprising contacting an effective number of recombinant adenovirus particles according to the invention with the cell under conditions where the recombinant adenovirus particles can transduce the cell and effect cytolysis thereof.

In another aspect, the present invention provides a pharmaceutical composition comprising a recombinant adenovirus particle according to the invention and a pharmaceutically acceptable carrier.

In another aspect, the present invention provides a method of treating a host organism having a neoplastic condition, comprising administering a therapeutically effective amount of the pharmaceutical composition according to the invention to the host organism. In one embodiment, the host organism is a human patient.

### BRIEF DESCRIPTION OF THE DRAWINGS

FIG. 1 depicts the vector genome of both Ar20-1004 and Ar20-1007 which express a mouse and a human GM-CSF, respectively. The adenoviral packaging signal is located 3′ to the LITR and 5′ to the pA (SV40 early). The E2F promoter is operatively linked to the E1 coding region. The GP19 coding sequence in the E3 region is deleted and the GM-CSF coding sequence is inserted in its place.

FIG. 2 depicts the vector genome of both Ar20-1006 and Ar20-1010 which express a human and a mouse GM-CSF, respectively. The adenoviral packaging signal is located 3′ to the LITR and 5′ to the pA (SV40 early). The TERT promoter is operatively linked to the E1 coding region. The GP19 coding sequence in the E3 region is deleted and the GM-CSF coding sequence is inserted in its place.

FIG. 3 shows the structure of some of the RCAs/rearranged vector detected in an assay that detects replication competent viruses in a preparation of selectively replicating virus. In this case the selectively replicating virus was Ar6pAE2fE3F as described in WO 02/067861 and PCT application PCT/US03/18243. The right end of the rearranged vector contains the packaging signal, suggesting recombination mechanisms of either intermolecular recombination or polymerase jumping. These RCAs had a deletion of some or all of E2F promoter, deletion of the p(A), duplication of part or all of E4 promoter and/or duplication of the packaging signal.

FIG. 4A-G shows the sequence for regions in Ar20-1007 confirmed by DNA sequencing. A-D) Nucleotides 1 through 2055 of Ar20-1007 (SEQ ID NO:4), containing ITR, packaging signal, poly A, E2F-1 promoter, E1a gene and a portion of the E1b gene. E-G) Nucleotides 28781 through 29952 of Ar20-1007 (SEQ ID NO:5) containing the E3-6.7 gene, the human GM-CSF cDNA and translated protein (SEQ ID NO:6) and the ADP gene.

FIG. 5A-B shows the sequence of a region of Ar20-1004 (SEQ ID NO:7) encoding for mouse GM-CSF. Single letter amino acid code underneath the corresponding nucleotides represents the derived protein sequence of mouse GM-CSF (SEQ ID NO:8).

FIG. 6 shows data that demonstrates anti-tumor efficacy in a Hep3B xenograft model injected with Ar20-1004 or Ar-20-1007. Nude mice bearing subcutaneous Hep3B tumors are injected intratumorally five times on the days indicated by the arrows. The group averages (+sem, n=10/group) are shown for mice that received 2×10⁶ vp (panel A), 2×10⁷ vp (panel B), or 2×10⁸ vp (panel C). *, indicates p<0.05 vs. HBSS treatment. +, indicates p<0.05 vs. dose-matched Addl312. #, indicates p<0.05 vs. dose-matched Addl1520. Symbols above the data points indicate significance for all groups below the symbols. Symbols below Ar20-1004 indicate significance for the Ar20-1004 group only. Statistical analysis was performed by Dunnett's method of ANOVA with either HBSS or Addl312 as the control group. Since there is no group of mice treated with 2×10⁶ vp of Addl312, the data for 2×10⁶ vp is compared to the more stringent Addl312 dose level of 2×10⁷ vp, as noted in the graph inset.

FIG. 7 shows anti-tumor efficacy in the PC3M-2Ac6 xenograft model injected with Ar20-1004 or Ar-20-1007. Nude mice bearing subcutaneous PC3M-2Ac6 tumors are injected intratumorally five times on the days indicated by the arrows. Saline or vector treatments are indicated in the graph insets. The group averages (+sem, n=10/group) are shown for mice that received 5×10⁷ vp (panel A), 5×10⁸ vp (panel B), or 5×10⁹ vp (panel C) per injection. *, indicates p<0.05 vs. HBSS treatment. +, indicates p<0.05 vs. dose-matched Addl312. #, indicates p<0.05 vs. dose-matched Addl1520. Symbols above the data points indicate significance for all groups below the symbols. Symbols below the data points indicate significance for the Ar20-1004 and Ar20-1007 groups only. Statistical analysis was performed by Dunnett's method of ANOVA with either HBSS or Addl312 as the control group. Similar results were obtained in a separate PC3M.2Ac6 experiment.

FIG. 8 shows anti-tumor efficacy in the LnCaP-FGC xenograft mode with Ar20-1004 or Ar-20-10071. SCID mice bearing subcutaneous LnCaP-FGC tumors are injected intratumorally five times on the days indicated by the arrows. Saline or vector treatments are indicated in the graph insets. The group average (+sem, n=8/group) are shown for mice that received 1×10⁸ vp (panel A), 1×10⁹ vp (panel B), or 1×10¹⁰ vp (panel C) per injection. *, indicates p<0.05 vs. HBSS treatment. +, indicates p<0.05 vs. dose-matched Addl312. Symbols above the data points indicate significance for all groups below the symbols. Symbols below Ar20-1004 indicate significance for the Ar20-1004 group only. Statistical analysis was performed by Dunnett's method of ANOVA with either HBSS or Addl312 as the control group.

FIG. 9A-F shows regions in Ar20-1006 confirmed by DNA sequencing. A-D) Nucleotides 1 through 2038 of Ar20-1006 (SEQ ID NO:9), containing ITR, packaging signal, poly A, hTERT promoter, E1a gene and a portion of the E1b gene. E-F) Nucleotides 28772 through 29671 of Ar20-1006 (SEQ ID NO:10) containing the E3-6.7 gene, the human GM-CSF cDNA and a portion of the ADP gene.

FIG. 10A-F shows regions in Ar20-1010 confirmed by DNA sequencing. A-D) Nucleotides 1 through 2041 of Ar20-1010 (SEQ ID NO:11), containing ITR, packaging signal, poly A, hTERT promoter, E1a gene and a portion of the E1b gene. E-F) Nucleotides 28781 through 29575 of Ar20-1010 (SEQ ID NO:12) containing the E3-6.7 gene, the mouse GM-CSF cDNA.

### DETAILED DESCRIPTION OF THE INVENTION

In describing the present invention, the following terms are employed and are intended to be defined as indicated below.

### DEFINITIONS

The terms “virus,” “viral particle,” “vector particle,” “viral vector particle,” and “virion” are used interchangeably and are to be understood broadly as meaning infectious viral particles that are formed when, e.g., a viral vector of the invention is transduced into an appropriate cell or cell line for the generation of infectious particles. Viral particles according to the invention may be utilized for the purpose of transferring DNA into cells either in vitro or in vivo. For purposes of the present invention, these terms refer to adenoviruses, including recombinant adenoviruses formed when an adenoviral vector of the invention is encapsulated in an adenovirus capsid.

As used herein, the terms “adenovirus” and “adenoviral particle” are used to include any and all viruses that may be categorized as an adenovirus, including any adenovirus that infects a human or an animal, including all groups, subgroups, and serotypes. Thus, as used herein, “adenovirus” and “adenovirus particle” refer to the virus itself or derivatives thereof and cover all serotypes and subtypes and both naturally occurring and recombinant forms, except where indicated otherwise. In one embodiment, such adenoviruses are ones that infect human cells. Such adenoviruses may be wildtype or may be modified in various ways known in the art or as disclosed herein. Such modifications include modifications to the adenovirus genome that is packaged in the particle in order to make an infectious virus. Such modifications include deletions known in the art, such as deletions in one or more of the E1a, E1b, E2a, E2b, E3, or E4 coding regions. The terms also include replication-conditional adenoviruses; that is, viruses that preferentially replicate in certain types of cells or tissues but to a lesser degree or not at all in other types. In one embodiment of the invention, the adenoviral particles selectively replicate in tumor cells and or abnormally proliferating tissue, such as solid tumors and other neoplasms. These include the viruses disclosed in U.S. Pat. Nos. 5,677,178, 5,698,443, 5,871,726, 5,801,029, 5,998,205, and 6,432,700, the disclosures of which are incorporated herein by reference in their entirety. Such viruses are sometimes referred to as “cytolytic” or “cytopathic” viruses (or vectors), and, if they have such an effect on neoplastic cells, are referred to as “oncolytic” viruses (or vectors).

The terms “vector,” “polynucleotide vector,” “polynucleotide vector construct,” “nucleic acid vector construct,” and “vector construct” are used interchangeably herein to mean any nucleic acid construct for gene transfer, as understood by one skilled in the art.

As used herein, the term “viral vector” is used according to its art-recognized meaning. It refers to a nucleic acid vector construct that includes at least one element of viral origin and may be packaged into a viral vector particle. The viral vector particles may be utilized for the purpose of transferring DNA, RNA or other nucleic acids into cells either in vitro or, in vivo. Viral vectors include, but are not limited to, retroviral vectors, vaccinia vectors, lentiviral vectors, herpes virus vectors (e.g., HSV), baculoviral vectors, cytomegalovirus (CMV) vectors, papillomavirus vectors, simian virus (SV40) vectors, Sindbis vectors, semliki forest virus vectors, phage vectors, adenoviral vectors, and adeno-associated viral (MV) vectors. Suitable viral vectors are described in U.S. Pat. Nos. 6,057,155, 5,543,328 and 5,756,086. For purposes of the present invention, the viral vector is preferably an adenoviral vector.

The terms “adenovirus vector” and “adenoviral vector” are used interchangeably and are well understood in the art to mean a polynucleotide comprising all or a portion of an adenovirus genome. An adenoviral vector of this invention may be in any of several forms, including, but not limited to, naked DNA, DNA encapsulated in an adenovirus capsid, DNA packaged in another viral or viral-like form (such as herpes simplex, and AAV), DNA encapsulated in liposomes, DNA complexed with polylysine, complexed with synthetic polycationic molecules, conjugated with transferrin, complexed with compounds such as PEG to immunologically “mask” the molecule and/or increase half-life, or conjugated to a non-viral protein.

In the context of adenoviral vectors, the term “5′” is used interchangeably with “upstream” and means in the direction of the left inverted terminal repeat (ITR). In the context of adenoviral vectors, the term “3′” is used interchangeably with “downstream” and means in the direction of the right ITR.

As used herein, the terms “cancer,” “cancer cells,” “neoplastic cells,” “neoplasia,” “tumor,” and “tumor cells” (used interchangeably) refer to cells that exhibit relatively autonomous growth, so that they exhibit an aberrant growth phenotype characterized by a significant loss of control of cell proliferation. Neoplastic cells can be malignant or benign.

The terms “coding sequence” and “coding region” refer to a nucleic acid sequence that is transcribed into RNA such as mRNA, rRNA, tRNA, snRNA, sense RNA or antisense RNA. In one embodiment, the RNA is then translated in a cell to produce a protein.

The terms “complement” and “complementary” refer to two nucleotide sequences that comprise antiparallel nucleotide sequences capable of pairing with one another upon formation of hydrogen bonds between the complementary base residues in the antiparallel nucleotide sequences.

The term “consists essentially of” as used herein with reference to a particular nucleotide sequence means that the particular sequence may have up to 20 additional residues on either the 5′ or 3′ end or both, wherein the additional residues do not materially affect the basic and novel characteristics of the recited sequence.

The term “enhancer” within the meaning of the invention may be any genetic element, e.g., a nucleotide sequence that increases transcription of a coding sequence operatively linked to a promoter to an extent greater than the transcription activation effected by the promoter itself when operatively linked to the coding sequence, i.e. it increases transcription from the promoter.

The term “expression” refers to the transcription and/or translation of an endogenous gene, transgene or coding region in a cell. In the case of an antisense construct, expression may refer to the transcription of the antisense DNA only.

The term “E2F promoter” refers to a native E2F promoter and functional fragments, mutations and derivatives thereof. The E2F promoter does not have to be the full-length wild type promoter. One skilled in the art knows how to derive fragments from an E2F promoter and test them for the desired selectivity. An E2F promoter fragment of the present invention has promoter activity selective for tumor cells, i.e. drives tumor selective expression of an operatively linked coding sequence. The term “tumor selective promoter activity” as used herein means that the promoter activity of a promoter fragment of the present invention in tumor cells is higher than in non-tumor cell types. In one embodiment, the E2F promoter of the invention is a mammalian E2F promoter. In one embodiment, the mammalian E2F promoter is a human E2F promoter. In one embodiment of the invention, the E2F promoter consists essentially of SEQ ID No:1

In other embodiments, a E2F promoter according to the present invention has at least 60%, at least 70%, at least 80%, at least 90%, at least 95%, at least 99%, or 100% identity to the sequence shown in SEQ ID NO:1, when compared and aligned for maximum correspondence, as measured using one of the following sequence comparison algorithms or by visual inspection. In one embodiment, the given % sequence identity exists over a region of the sequences that is at least about 50 nucleotides in length. In another embodiment, the given % sequence identity exists over a region of at least about 100 nucleotide. In another embodiment, the given % sequence identity exists over a region of at least about 200 nucleotides. In another embodiment, the given % sequence identity exists over the entire length of the sequence.

The term “TERT promoter” refers to a native TERT promoter and functional fragments, mutations and derivatives thereof. The TERT promoter does not have to be the full-length wild type promoter. One skilled in the art knows how to derive fragments from a TERT promoter and test them for the desired selectivity. A TERT promoter fragment of the present invention has promoter activity selective for tumor cells, i.e. drives tumor selective expression of an operatively linked coding sequence. In one embodiment, the TERT promoter of the invention is a mammalian TERT promoter. In one embodiment, the mammalian TERT promoter is a human TERT promoter.

In one embodiment of the invention, the TERT promoter consists essentially of SEQ ID No:2 or SEQ ID NO:3

In other embodiments, an TERT promoter according to the present invention has at least 60%, at least 70%, at least 80%, at least 90%, at least 95%, at least 99%, or 100% identity to the sequence shown in SEQ ID NO:2 or SEQ ID NO:3, when compared and aligned for maximum correspondence, as measured using one of the following sequence comparison algorithms or by visual inspection. In one embodiment, the given % sequence identity exists over a region of the sequences that is at least about 50 nucleotides in length. In another embodiment, the given % sequence identity exists over a region of at least about 100 nucleotide. In another embodiment, the given % sequence identity exists over a region of at least about 200 nucleotides. In another embodiment, the given % sequence identity exists over the entire length of the sequence.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), by the BLAST algorithm, Altschul et al., J. Mol. Biol. 215: 403-410 (1990), with software that is publicly available through the National Center for Biotechnology Information, or by visual inspection (see generally, Ausubel et al., infra). For purposes of the present invention, optimal alignment of sequences for comparison is most preferably conducted by the local homology algorithm of Smith & Waterman, Adv. Appl. Math. 2: 482 (1981).

In one embodiment, an E2F promoter according to the present invention has a full-length complement that hybridizes to the sequence shown in SEQ ID NO:1 under stringent conditions. In another embodiment, the TERT promoter according to the present invention has a full-length complement that hybridizes to the sequence shown in SEQ ID NO:2 and/or SEQ ID NO:3 under stringent conditions. The phrase “hybridizing to” refers to the binding, duplexing, or hybridizing of a molecule to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent wash conditions” in the context of nucleic acid hybridization experiments such as Southern and Northern hybridizations are sequence dependent, and are different under different environmental parameters. Longer sequences hybridize at higher temperatures. An extensive guide to the hybridization of nucleic acids is found in Tijssen (1993) Laboratory Techniques in Biochemistry and Molecular Biology-Hybridization with Nucleic Acid Probes part 1 chapter 2 “Overview of principles of hybridization and the strategy of nucleic acid probe assays” Elsevier, New York. Generally, highly stringent hybridization and wash conditions are selected to be about 5° C. to 20° C. (preferably 5° C.) lower than the thermal melting point (Tₘ) for the specific sequence at a defined ionic strength and pH. Typically, under highly stringent conditions a probe will hybridize to its target subsequence, but to no other unrelated sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids that have more than 100 complementary residues on a filter in a Southern or northern blot is 50% formamide with 1 mg of heparin at 42° C., with the hybridization being carried out overnight. An example of highly stringent wash conditions is 0.15M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization.

The term “gene” refers to a defined region that is located within a genome and that, in addition to the aforementioned coding sequence, comprises other, primarily regulatory, nucleic acid sequences responsible for the control of expression, i.e., transcription and translation of the coding portion. A gene may also comprise other 5′ and 3′ untranslated sequences and termination sequences. Depending on the source of the gene, further elements that may be present are, for example, introns.

The term “gene essential for replication” refers to a nucleic acid sequence whose transcription is required for a viral vector to replicate in a target cell. For example, in an adenoviral vector of the invention, a gene essential for replication may be selected from the group consisting of the E1a, E1b, E2a, E2b, and E4 genes.

The terms “heterologous” and “exogenous” as used herein with reference to nucleic acid molecules such as promoters and gene coding sequences, refer to sequences that originate from a source foreign to a particular virus or host cell or, if from the same source, are modified from their original form. Thus, a heterologous gene in a virus or cell includes a gene that is endogenous to the particular virus or cell but has been modified through, for example, codon optimization. The terms also include non-naturally occurring multiple copies of a naturally occurring nucleic acid sequence. Thus, the terms refer to a nucleic acid segment that is foreign or heterologous to the virus or cell, or homologous to the virus or cell but in a position within the host viral or cellular genome in which it is not ordinarily found.

The term “homologous” as used herein with reference to a nucleic acid molecule refers to a nucleic acid sequence naturally associated with a host virus or cell.

The terms “identical” or percent “identity” in the context of two or more nucleic acid or protein sequences, refer to two or more sequences or subsequences that are the same or have a specified percentage of amino acid residues or nucleotides that are the same, when compared and aligned for maximum correspondence, as measured using one of the sequence comparison algorithms described herein, e.g. the Smith-Waterman algorithm, or by visual inspection.

In the context of the present invention, the term “isolated” refers to a nucleic acid molecule, polypeptide, virus, or cell that, by the hand of man, exists apart from its native environment and is therefore not a product of nature. An isolated nucleic acid molecule or polypeptide may exist in a purified form or may exist in a non-native environment such as, for example, a recombinant host cell. An isolated virus or cell may exist in a purified form, such as in a cell culture, or may exist in a non-native environment such as, for example, a recombinant or xenogeneic organism.

The term “native” refers to a gene that is present in the genome of the wildtype virus or cell.

The term “naturally occurring” or “wildtype” is used to describe an object that can be found in nature as distinct from being artificially produced by man. For example, a protein or nucleotide sequence present in an organism (including a virus), which can be isolated from a source in nature and which has not been intentionally modified by man in the laboratory, is naturally occurring.

The term “nucleic acid” refers to deoxyribonucleotides or ribonucleotides and polymers thereof (“polynucleotides”) in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid molecule/polynucleotide also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19: 5081 (1991); Ohtsuka et al., J. Biol. Chem. 260: 2605-2608 (1985); Rossolini et al., Mol. Cell. Probes 8: 91-98 (1994)). Nucleotides are indicated by their bases by the following standard abbreviations: adenine (A), cytosine (C), thymine (T), and guanine (G).

A nucleic acid sequence is “operatively linked” when it is placed into a functional relationship with another nucleic acid sequence. For example, a promoter or regulatory DNA sequence is said to be “operatively linked” to a DNA sequence that codes for an RNA or a protein if the two sequences are operatively linked, or situated such that the promoter or regulatory DNA sequence affects the expression level of the coding or structural DNA sequence. Operatively linked DNA sequences are typically, but not necessarily, contiguous.

The term “ORF” means Open Reading Frame.

As used herein, a “packaging cell” is a cell that is able to package adenoviral genomes or modified genomes to produce viral particles. It can provide a missing gene product or its equivalent. Thus, packaging cells can provide complementing functions for the genes deleted in an adenoviral genome and are able to package the adenoviral genomes into the adenovirus particle. The production of such particles requires that the genome be replicated and that those proteins necessary for assembling an infectious virus are produced. The particles also can require certain proteins necessary for the maturation of the viral particle. Such proteins can be provided by the vector or by the packaging cell.

The term “promoter” refers to an untranslated DNA sequence usually located upstream of the coding region that contains the binding site for RNA polymerase II and initiates transcription of the DNA. The promoter region may also include other elements that act as regulators of gene expression. The term “minimal promoter” refers to a promoter element, particularly a TATA element that is inactive or has greatly reduced promoter activity in the absence of upstream activation elements.

The term “recombinant” as used herein with reference to nucleic acid molecules refers to a combination of nucleic acid molecules that are joined together using recombinant DNA technology into a progeny nucleic acid molecule. As used herein with reference to viruses, cells, and organisms, the terms “recombinant,” “transformed,” and “transgenic” refer to a host virus, cell, or organism into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Recombinant viruses, cells, and organisms are understood to encompass not only the end product of a transformation process, but also recombinant progeny thereof. A “non-transformed,” “non-transgenic,” or “non-recombinant” host refers to a wildtype virus, cell, or organism that does not contain the heterologous nucleic acid molecule.

“Regulatory elements” are sequences involved in controlling the expression of a nucleotide sequence. Regulatory elements include promoters, enhancers, and termination signals. They also typically encompass sequences required for proper translation of the nucleotide sequence.

A “selectable marker gene” is a gene whose expression in a cell gives the cell a selective advantage. The selective advantage possessed by the cells transformed with the selectable marker gene may be due to their ability to grow in the presence of a negative selective agent, such as an antibiotic, compared to the growth of non-transformed cells. The selective advantage possessed by the transformed cells, compared to non-transformed cells, may also be due to their enhanced or novel capacity to utilize an added compound as a nutrient, growth factor or energy source.

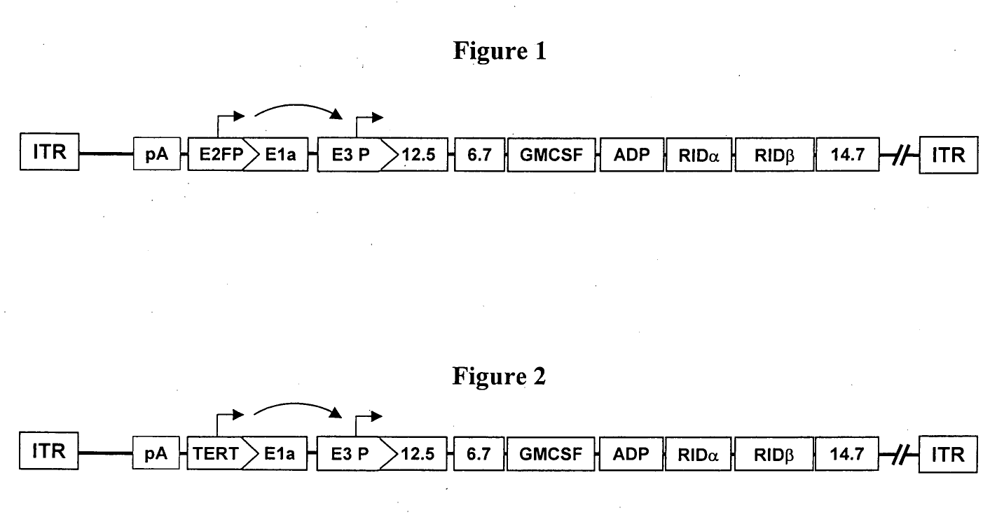

A “termination signal sequence” within the meaning of the invention may be any genetic element that causes RNA polymerase to terminate transcription, such as for example a polyadenylation signal sequence. A polyadenylation signal sequence is a recognition region necessary for endonuclease cleavage of an RNA transcript that is followed by the polyadenylation consensus sequence AATAAA. A polyadenylation signal sequence provides a “polyA site”, i.e. a site on a RNA transcript to which adenine residues will be added by post-transcriptional polyadenylation. Polyadenylation signal sequences are useful insulating sequences for transcription units within eukaryotic cells and eukaryotic viruses. Generally, the polyadenylation signal sequence includes a core poly(A) signal that consists of two recognition elements flanking a cleavage-polyadenylation site (e.g., FIG. 1 of WO 02/067861 and WO 02/068627). Typically, an almost invariant AAUAAA hexamer lies 20 to 50 nucleotides upstream of a more variable element rich in U or GU residues. Cleavage between these two elements is usually on the 3′ side of an A residue and, in vitro, is mediated by a large, multicomponent protein complex. The choice of a suitable polyadenylation signal sequence will consider the strength of the polyadenylation signal sequence, as completion of polyadenylation process correlates with poly(A) site strength (Chao et al., Molecular and Cellular Biology, 1999, 19:5588-5600). For example, the strong SV40 late poly(A) site is committed to cleavage more rapidly than the weaker SV40 early poly(A) site. The person skilled in the art will consider to choose a stronger polyadenylation signal sequence if a more substantive reduction of nonspecific transcription is required in a particular vector construct. In principle, any polyadenylation signal sequence may be useful for the purposes of the present invention. However, in preferred embodiments of this invention the termination signal sequence is either the SV40 late polyadenylation signal sequence or the SV40 early polyadenylation signal sequence. In one embodiment of the invention, the termination signal sequence is isolated from its genetic source and inserted into the viral vector at a suitable position upstream of an E2F or TERT promoter.

The term “HeLa-S3” means the human cervical tumor-derived cell line available from American Type Culture Collection (ATCC, Manassas, Va.) and designated as ATCC number CCL-2.2. HeLa-S3 is a clonal derivative of the parent HeLa line (ATCC CCL-2). HeLa-S3 was cloned in 1955 by T. T. Puck et al. (J. Exp. Med. 103: 273-284 (1956)).

### Adenoviral Vectors of the Invention

The present invention provides novel adenoviral vectors based on the oncolytic adenoviral vector strategy as described in WO 96/17053 and WO 99/25860. In particular, oncolytic adenoviral vectors are disclosed in which expression of an adenoviral gene, which is essential for replication, is controlled by a regulatory region that is selectively transactivated in cancer cells. In accordance with the present invention, such a cancer selective regulatory region is an E2F or TERT promoter described in further detail herein. The invention further comprises adenoviral vector particles, which comprise the viral vectors of the invention.

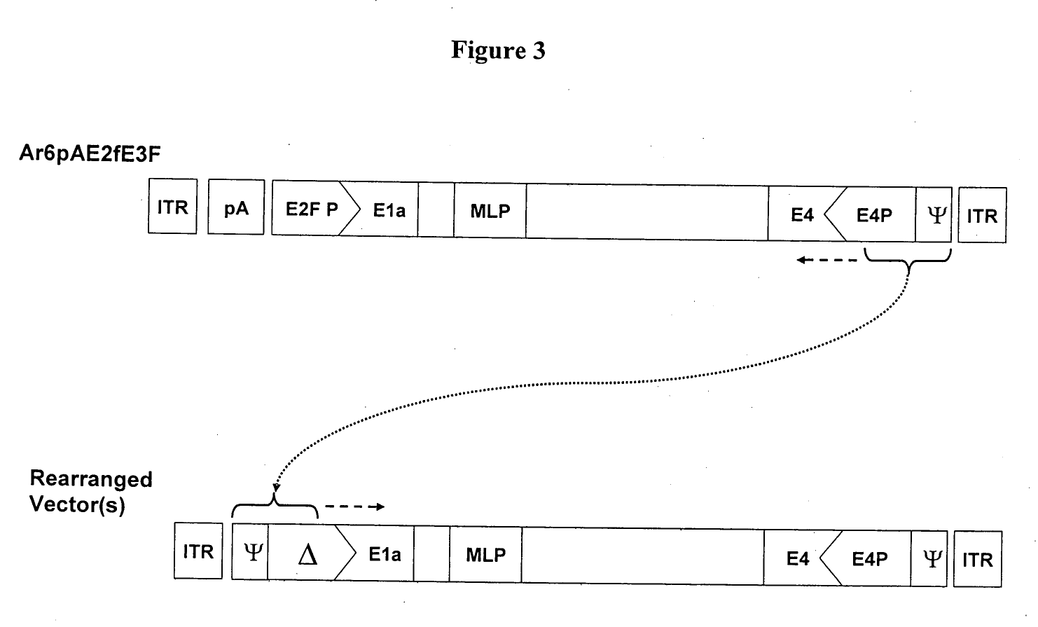

The viral vectors and particles of the present invention with an E2F promoter operably linked to a gene essential for replication are similar to those disclosed in PCT publication WO 02/067861 and Bristol et al. (“In vitro and in vivo activities of an oncolytic adenoviral vector designed to express GM-CSF” Mol. Ther. 2003 June; 7(6):755-64). Vectors described in WO 02/067861 and Bristol et al. (2003) have an adenoviral packaging signal located on the right end, 3′ of the E4 region and 5′ of the right ITR(RITR). The viral vectors of the present invention have the adenoviral packaging located 3′ of the left ITR (LITR) and 5′ of the E1a coding sequences. In one embodiment, the packaging signal in the vectors of the present invention is located 3′ of the LITR and 5′ of the termination signal sequence. The Ar6pAE2fE3F vector is described in PCT publication WO 02/067861 and PCT International Application, filed Jun. 9, 2003 titled “Assay to detect replication competent viruses”. This International Application describes a biological assay to detect replication competent virus (RCV) in replication selective virus (a.k.a. selectively replicating; e.g., oncolytic virus) preparations. It also describes the detection of an RCV in a preparation of Ar6pAE2fE3F and further describes a hypothesis for how the detected RCVs are created through recombination events. These recombination events are believed to be non-homologous recombination events. The most prevalent “class” of detected recombinants from Ar6pAE2fE3F involves a recombination event that duplicated part of the right end of the adenoviral vector and inserted this copy in place of part of the left end of the vector. See FIG. 3. Thus this recombinant contained a packaging signal on each side of the adenoviral vector genome. As a result, the rearranged viruses lost their tumor selectivity, grew preferentially on nontransformed MRC-5 cells and were more cytotoxic to primary and nontransformed cells than the wild type Ad5 virus. Interestingly, this type of RCV was not detected in all of the replication selective preparations where the adenoviral vector contained a packaging signal adjacent to the RITR. This indicates the recombination events are specific for each vector and can not be readily predicted.

It is hypothesized that the creation and propagation of the above-described type of RCV is inhibited or prevented in preparations of adenoviral vectors of the present invention, Ar20-1004, Ar20-1006, Ar20-1007 and Ar20-1010, because if the right end of these vectors is duplicated from the right end and inserted on the left end of the virus, this will result in the recombined vector not containing the packaging signal. Therefore, such a recombinant virus will not be propagated and/or amplified through subsequent passaging of the virus.

The adenoviral particles of the invention are made by standard techniques known to those skilled in the art. Adenoviral vectors are transferred into packaging cells by techniques known to those skilled in the art. Packaging cells typically complement any functions deleted from the wildtype adenoviral genome. The production of such particles requires that the vector be replicated and that those proteins necessary for assembling an infectious virus be produced. The packaging cells are cultured under conditions that permit the production of the desired viral vector particle. The particles are recovered by standard techniques. The preferred packaging cells are those that have been designed to limit homologous recombination that could lead to wildtype adenoviral particles. Cells that may be used to produce the adenoviral particles of the invention include the human embryonic kidney cell line 293 (Graham et al., J. Gen. Virol. 36:59-72 (1977)), the human embryonic retinoblast cell line PER.C6 (U.S. Pat. Nos. 5,994,128 and 6,033,908; Fallaux et al., Hum. Gene Ther. 9: 1909-1917 (1998)), and the human cervical tumor-derived cell line HeLa-S3 (U.S. patent application 60/463,143; ATCC #CCL-2.2).

The present invention contemplates the use of all adenoviral serotypes to construct the oncolytic vectors and virus particles according to the present invention. For example, the adenoviral nucleic acid backbone is derived from adenovirus serotype 2(Ad2), 5 (Ad5) or 35 (Ad35), although other serotype adenoviral vectors can be employed. Adenoviral stocks that can be employed according to the invention include any adenovirus serotype. Adenovirus serotypes 1 through 47 are currently available from American Type Culture Collection (ATCC, Manassas, Va.), and the invention includes any other serotype of adenovirus available from any source including those serotypes listed in Table 1. The adenoviruses that can be employed according to the invention may be of human or non-human origin, such as bovine, porcine, canine, simian, avian. For instance, an adenovirus can be of subgroup A (e.g., serotypes 12, 18, 31), subgroup B (e.g., serotypes 3, 7, 11, 14, 16, 21, 34, 35, 50), subgroup C (e.g., serotypes 1, 2, 5, 6), subgroup D (e.g., serotypes 8, 9, 10, 13, 15, 17, 19, 20, 22-30, 32, 33, 36-39, 42, 47, 49, 51), subgroup E (serotype 4), subgroup F (serotype 40,41), or any other adenoviral serotype.

TABLE 1
Examples Of Human And Animal Adenoviruses Including The American Type Culture Collection
Catalog # For A Representative Virus Of The Respective Classification

| | |
|---|---|
| Adenovirus Type 21 ATCC VR-1099 | SA18 (Simian adenovirus 18) ATCC VR-943 |
| SA17 (Simian adenovirus 17) ATCC VR-942 | Adenovirus Type 47 ATCC VR-1309 |
| Adenovirus Type 44 ATCC VR-1306 | Avian adenovirus Type 4 ATCC VR-829 |
| Avian adenovirus Type 5 ATCC VR-830 | Avian adenovirus Type 7 ATCC VR-832 |
| Avian adenovirus Type 8 ATCC VR-833 | Avian adenovirus Type 9 ATCC VR-834 |
| Avian adenovirus Type 10 ATCC VR-835 | Avian adenovirus Type 2 ATCC VR-827 |
| Adenovirus Type 45 ATCC VR-1307 | Adenovirus Type 38 ATCC VR-988 |
| Adenovirus Type 46 ATCC VR-1308 | Simian adenovirus ATCC VR-541 |
| SA7 (Simian adenovirus 16) ATCC VR-941 | Frog adenovirus (FAV-1) ATCC VR-896 |
| Adenovirus type 48 (candidate) ATCC VR-1406 | Adenovirus Type 42 ATCC VR-1304 |
| Adenovirus Type 49 (candidate) ATCC VR-1407 | Adenovirus Type 43 ATCC VR-1305 |
| Avian adenovirus Type 6 ATCC VR-831 | Avian adenovirus Type 3 |
| Bovine adenovirus Type 3 ATCC VR-639 | Bovine adenovirus Type 6 ATCC VR-642 |
| Canine adenovirus ATCC VR-800 | Bovine adenovirus Type 5 ATCC VR-641 |
| Adenovirus Type 36 ATCC VR-913 | Ovine adenovirus type 5 ATCC VR-1343 |
| Adenovirus Type 29 ATCC VR-272 | Swine adenovirus ATCC VR-359 |
| Bovine adenovirus Type 4 ATCC VR-640 | Bovine adenovirus Type 8 ATCC VR-769 |
| Bovine adenovirus Type 7 ATCC VR-768 | Adeno-associated virus Type2 (AAV-2H) |
| | ATCC VR-680 |
| Adenovirus Type 4 ATCC VR-4 | Adeno-associated virus Type3 (AAV-3H) |
| | ATCC VR-681 |
| Peromyscus adenovirus ATCC VR-528 | Adenovirus Type 15 ATCC VR-661 |
| Adenovirus Type 20 ATCC VR-662 | Chimpanzee adenovirus ATCC VR-593 |
| Adenovirus Type 31 ATCC VR-357 | Adenovirus Type 25 ATCC VR-223 |
| Chimpanzee adenovirus ATCC VR-592 | Chimpanzee adenovirus ATCC VR-591 |
| Adenovirus Type 26 ATCC VR-224 | Adenovirus Type 19 ATCC VR-254 |
| Adenovirus Type 23 ATCC VR-258 | Adenovirus Type 28 ATCC VR-226 |
| Adenovirus Type 6 ATCC VR-6 | Adenovirus Type 2 Antiserum: ATCC VR-1079 |
| Adenovirus Type 6 ATCC VR-1083 | Ovine adenovirus Type 6 ATCC VR-1340 |
| Adenovirus Type 3 ATCC VR-847 | Adenovirus Type 7 ATCC VR-7 |
| Adenovirus Type 39 ATCC VR-932 | Adenovirus Type 3 ATCC VR-3 |
| Bovine adenovirus Type 1 ATCC VR-313 | Adenovirus Type 14 ATCC VR-15 |
| Adenovirus Type 1 ATCC VR-1078 | Adenovirus Type 21 ATCC VR-256 |
| Adenovirus Type 18 ATCC VR-1095 | Baboon adenovirus ATCC VR-275 |
| Adenovirus Type 10 ATCC VR-11 | Adenovirus Type 33 ATCC VR-626 |
| Adenovirus Type 34 ATCC VR-716 | Adenovirus Type 15 ATCC VR-16 |
| Adenovirus Type 22 ATCC VR-257 | Adenovirus Type 24 ATCC VR-259 |
| Adenovirus Type 17 ATCC VR-1094 | Adenovirus Type 4 ATCC VR-1081 |
| Adenovirus Type 16 ATCC VR-17 | Adenovirus Type 17 ATCC VR-18 |
| Adenovirus Type 16 ATCC VR-1093 | Bovine adenovirus Type 2 ATCC VR-314 |
| SV-30 ATCC VR-203 | Adenovirus Type 32 ATCC VR-625 |
| Adenovirus Type 20 ATCC VR-255 | Adenovirus Type 13 ATCC VR-14 |
| Adenovirus Type 14 ATCC VR-1091 | Adenovirus Type 18 ATCC VR-19 |
| SV-39 ATCC VR-353 | Adenovirus Type 11 ATCC VR-849 |
| Duck adenovirus (Egg drop syndrome) ATCC | Adenovirus Type 1 ATCC VR-1 |
| VR-921 | |
| Chimpanzee adenovirus ATCC VR-594 | Adenovirus Type 15 ATCC VR-1092 |
| Adenovirus Type 13 ATCC VR-1090 | Adenovirus Type 8 ATCC VR-1368 |
| SV-31 ATCC VR-204 | Adenovirus Type 9 ATCC VR-1086 |
| Mouse adenovirus ATCC VR-550 | Adenovirus Type 9 ATCC VR-10 |
| Adenovirus Type 41 ATCC VR-930 | C1 ATCC VR-20 |
| Adenovirus Type 40 ATCC VR-931 | Adenovirus Type 37 ATCC VR-929 |
| Marble spleen disease virus | Adenovirus Type 35 ATCC VR-718 |
| SV-32 (M3) ATCC VR-205 | Adenovirus Type 28 ATCC VR-1106 |
| Adenovirus Type 10 ATCC VR-1087 | Adenovirus Type 20 ATCC VR-1097 |
| Adenovirus Type 21 ATCC VR-1098 | Adenovirus Type 25 ATCC VR-1103 |
| Adenovirus Type 26 ATCC VR-1104 | Adenovirus Type 31 ATCC VR-1109 |
| Adenovirus Type 19 ATCC VR-1096 | SV-36 ATCC VR-208 |
| SV-38 ATCC VR-355 | SV-25 (M8) ATCC VR-201 |
| SV-15 (M4) ATCC VR-197 | Adenovirus Type 22 ATCC VR-1100 |
| SV-23 (M2) ATCC VR-200 | Adenovirus Type 11 ATCC VR-12 |
| Adenovirus Type 24 ATCC VR-1102 | Avian adenovirus Type 1 |
| SV-11 (M5) ATCC VR-196 | Adenovirus Type 5 ATCC VR-5 |
| Adenovirus Type 23 ATCC VR-1101 | SV-27 (M9) ATCC VR-202 |
| Avian adenovirus Type 2 (GAL) ATCC VR-280 | SV-1 (M1) ATCC VR-195 |
| SV-17 (M6) ATCC VR-198 | Adenovirus Type 29 ATCC VR-1107 |
| Adenovirus Type 2 ATCC VR-846 | SV-34 ATCC VR-207 |
| SV-20 (M7) ATCC VR-199 | SV-37 ATCC VR-209 |
| SV-33 (M10) ATCC VR-206 | Avian adeno-associated virus ATCC VR-865 |
| Adeno-associated (satellite) virus Type 4 | Adenovirus Type 30 ATCC VR-273 |
| ATCC VR-646 | |
| Adeno-associated (satellite) virus Type1 | Infectious canine hepatitis (Rubarth's disease) |
| ATCCVR-645 | |
| Adenovirus Type 27 ATCC VR-1105 | Adenovirus Type 12 ATCC VR-863 |
| Adeno-associated virus Type 2 | Adenovirus Type 7a ATCC VR-848 |

The recombinant adenoviral vectors of this invention are useful in therapeutic treatment regimens for cancer. The vectors of the invention preferentially kill tumor cells. In one embodiment, the vectors of the invention, with an E2F promoter operably linked to a gene essential to replication, preferentially kill Rb-pathway defective tumor cells as compared to cells which are non-defective in the Rb-pathway. In another embodiment, the vectors of the invention, with a TERT promoter operably linked to a gene essential to replication, preferentially kill tumor cells with up-regulated expression of telomerase as compared to non-tumor cells. Without wishing to be limited by theoretical considerations, the specific regulation of viral replication by an E2F or TERT promoter, which is, in one embodiment, shielded from read-through transcription by the upstream termination signal sequence, avoids toxicity that would occur if the virus replicated in non-target tissues, allowing for a favorable efficacy/toxicity profile. Thus, the combination and the sequential positioning of the genetic elements employed in the vectors of this invention provide for and enhance the vector's selectivity, while at the same time synergistically minimizing toxicity and side effects in an animal. The recombinant viral vectors of the invention may further comprise a selective promoter linked to an adenoviral early gene, e.g., the E1A, E1B, E2 or E4 gene.

Without being bound by theory, the inventors believe that the mechanism of action is as follows. The selectivity of E2F-responsive promoters (hereinafter sometimes referred to as E2F promoters) is based on the derepression of the E2F promoter/transactivator in Rb-pathway defective tumor cells. In quiescent cells, E2F binds to the tumor suppressor protein pRB in ternary complexes. In its complexed form, E2F functions to repress transcriptional activity from promoters with E2F binding sites, including the E2F-1 promoter itself (Zwicker J, and Muller R. Cell cycle-regulated transcription in mammalian cells. Prog. Cell Cycle Res 1995; 1:91-99). Thus the E2F-1 promoter is transcriptionally inactive in resting cells. In normal cycling cells, pRB-E2F complexes are dissociated in a regulated fashion, allowing for controlled derepression of E2F and subsequent cell cycling (Dyson, N. The regulation of E2F by pRB-family proteins. Genes and Development 1998; 12:2245-2262).

In the majority of tumor types, the Rb cell cycle regulatory pathway is disrupted, suggesting that Rb-pathway deregulation is obligatory for tumorigenesis (Strauss M, Lukass J and Bartek J. Unrestricted cell cycling and cancer. Nat Med 1995; 12:1245-1246). These mutations can be in Rb itself or in other factors that have an effect on upstream regulators of pRB, such as the cyclin-dependent kinase, p16 (Weinberg, R A. The retinoblastoma protein and cell cycle control. Cell 1995; 81:323-330). One consequence of these mutations is the disruption of E2F-pRB binding and an increase in free E2F in tumor cells. The abundance of free E2F in turn results in high level expression of E2F responsive genes in tumor cells, driving them into S phase. The E2F-1 promoter used here has been shown to up-regulate the expression of marker genes in an adenovirus vector in a rodent tumor model but not normal proliferating cells in vivo (Parr M J et al. Tumor-selective transgene expression in vivo mediated by an E2F-responsive adenoviral vector. Nature Med 1997; October; 3(10):1145-1149).

An E2F-responsive promoter has at least one E2F binding site. In one embodiment, the E2F-responsive promoter is a mammalian E2F promoter. In one embodiment it is a human E2F promoter. For example, the E2F promoter may be the human E2F-1 promoter. Further, the human E2F-1 promoter may be, for example, a human E2F-1 promoter having the sequence as described in SEQ ID NO:1.

The E2F-responsive promoter does not have to be the full length wild type promoter, but should have a tumor-selectivity of at least 3-fold, at least 10-fold, at least 30-fold or even at least 300-fold. Tumor-selectivity can be determined by a number of assays using known techniques, such as the techniques employed in WO 02/067861, example 4, for example RT-PCR. In one embodiment, the tumor-selectivity of the adenoviral vectors can also be quantified by E1A RNA levels, as further described in WO 02/067861, example 4, and the E1A RNA levels obtained in H460 (ATCC, Cat. # HTB-177) cells can be compared to those in PrEC-(Clonetics Cat. #CC2555) cells in order to determine tumor-selectivity for the purposes of this invention. The relevant conditions of the experiment preferably follow those described in WO 02/067861. For example, Ar6pAE2fF in example 4 of WO 02/067861 displays a tumor-selectivity of 2665/8-fold, i.e. about 332-fold.

E2F responsive promoters typically share common features such as Sp I and/or ATT7 sites in proximity to their E2F site(s), which are frequently located near the transcription start site, and lack of a recognizable TATA box. E2F-responsive promoters include E2F promoters such as the E2F-1 promoter, dihydrofolate reductase (DHFR) promoter, DNA polymerase A (DPA) promoter, c-myc promoter and the B-myb promoter. The E2F-1 promoter contains four E2F sites that act as transcriptional repressor elements in serum-starved cells. In one embodiment, an E2F-responsive promoter has at least two E2F sites.

Without being bound by theory, the understanding of selective TERT expression in cancer is based on the current knowledge of the molecular underpinnings involved in tumorigenesis. TERT is the rate-limiting catalytic subunit of telomerase, a multicomponent ribonucleoprotein enzyme that has also been shown to be active in ˜85% of human cancers but not normal somatic cells (Kilian A et al. Hum Mol. Genet. 1997 November; 6(12):2011-9; Kim N W et al. Science. 1994 Dec. 23; 266(5193):2011-5; Shay J W et al. European Journal of Cancer 1997; 5, 787-791; Stewart S A et al. Semin Cancer Biol. 2000 December; 10(6):399406). Telomerase synthesizes telomeric DNA to enable cells to proliferate without senescence. In humans this activity is restricted to germ line cells, stem cells, and activated B and T cells, an attribute necessary for these cells to repopulate diminished cell populations or mediate an immune response (Kim N W et al. Science. 1994 Dec. 23; 266(5193):2011-5; Hiyama K et al. J Natl Cancer Inst. 1995 Jun. 21; 87(12):895-902). However, most other normal human cells have a limited lifespan due to lack of telomerase (Poole J C et al. Gene. 2001 May 16; 269(1-2):1-12; Shay J W et al. Hum Mol Genet. 2001 April; 10(7):677-85). Cancer cells appear to require immortalization for tumorigenesis and telomerase activity is almost always necessary for immortalization (Kim N W et al. Science. 1994 Dec. 23; 266(5193):2011-5; Kiyono T et al. Nature, 1998; 396:84), although there is an alternative pathway not involving telomerase that maintains telomere length in a small percentage of tumors (Bryan T M et al. Nat. Med. 1997 November; 3(11):1271-4). Thus, the majority of tumors have both a disregulated telomerase pathway specifically targeted by viruses of the invention utilizing a TERT promoter operably linked to a gene and/or coding region essential for replication (e.g. E1a).

The term TERT promoter refers to a native TERT promoter and functional fragments, mutations and derivatives thereof. The TERT promoter does not have to be a full-length wild type promoter. One skilled in the art knows how to derive fragments from a TERT promoter and test them for the desired specificity. In one embodiment, a TERT promoter of the invention is a mammalian TERT promoter. In a further embodiment the mammalian TERT promoter, is a human TERT promoter (hTERT). In one embodiment of the invention, the TERT promoter consists essentially of SEQ ID NO:2 which is a 397 bp fragment of the hTERT promoter. In another embodiment of the invention, the TERT promoter consists essentially of SEQ ID NO:3, which is a 245 bp fragment of the hTERT promoter. In one embodiment, a TERT promoter is operably linked to the adenovirus E1A, E1B, E2 or E4 region.

A recombinant viral vector of the invention may further comprise a termination signal sequence. The termination signal sequence increases the therapeutic effect because it reduces replication and toxicity of the oncolytic adenoviral vectors in non-target cells. Oncolytic vectors of the present invention that have a polyadenylation signal inserted upstream of the E1a coding region have been shown may be superior to their non-modified counterparts as they have demonstrated the lowest level of E1a expression in nontarget cells. Thus, insertion of a polyadenylation signal sequence to stop nonspecific transcription from the left ITR may improve the specificity of E1a expression from the respective promoter. Insertion of the polyadenylation signal sequences may therefore reduce replication of the oncolytic adenoviral vector in nontarget cells and therefore reduce toxicity. A termination signal sequence may also be placed upstream of (5′ to) any promoter in the vector. In one embodiment, the terminal signal sequence is placed 5′ to the E2F promoter which is operatively linked to the E1a coding sequences. In another embodiment, the terminal signal sequence is placed 5′ to the TERT promoter which is operatively linked to the E1a coding sequence.

In an alternative embodiment, the invention further comprises a mutation or deletion in the E1b gene. In one embodiment, the mutation or deletion in the E1b gene is such that the E1b-19 kD protein becomes non-functional. This modification of the E1b region may be included in vectors where all or a part of the E3 region is present.

### Transgenes

The vectors of the invention may include one or more transgenes. In this way, various genetic capabilities may be introduced into target cells. In one embodiment, the transgene encodes a selectable marker. In another embodiment, the transgene encodes a cytotoxic protein. These vectors encoding a cytotoxic protein may be used to eliminate certain cells in either an investigational setting or to achieve a therapeutic effect. For example, in certain instances, it may be desirable to enhance the degree of therapeutic efficacy by enhancing the rate of cytotoxic activity. This could be accomplished by coupling the cell-specific replicative cytotoxic activity with expression of, one or more metabolic enzymes such as HSV-tk, nitroreductase, cytochrome P450 or cytosine deaminase (CD) which render cells capable of metabolizing 5-fluorocytosine (5-FC) to the chemotherapeutic agent 5-fluorouracil (5-FU), carboxylesterase (CA), deoxycytidine kinase (dCK), purine nucleoside phosphorylase (PNP), carboxypeptidase G2 (CPG2; Niculescu-Duvaz et al. J Med. Chem. 2004 May 6; 47(10):2651-2658), thymidine phosphorylase (TP), thymidine kinase (TK), xanthine-guanine phosphoribosyl transferase (XGPRT) or a drug activator, such as B2, B5 and B10 (ZGene) designed to work with drugs such as gemcitabine, cladribine, and fludarabine. This type of transgene may also be used to confer a bystander effect.

Additional transgenes that may be introduced into a vector of the invention include a factor capable of initiating apoptosis, antisense or ribozymes, which among other capabilities may be directed to mRNAs encoding proteins essential for proliferation of the cells or a pathogen, such as structural proteins, transcription factors, polymerases, etc., viral or other pathogenic proteins, where the pathogen proliferates intracellularly, cytotoxic proteins, e.g., the chains of diphtheria, ricin, abrin, etc., genes that encode an engineered cytoplasmic variant of a nuclease (e.g., RNase A) or protease (e.g., trypsin, papain, proteinase K, carboxypeptidase, etc.), chemokines, such as MCP3 alpha or MIP-1, pore-forming proteins derived from viruses, bacteria, or mammalian cells, fusgenic genes, chemotherapy sensitizing genes and radiation sensitizing genes. Other genes of interest include cytokines, antigens, transmembrane proteins, and the like, such as IL-1, IL-2, IL-4, IL-5, IL-6, IL-10, IL-12, IL-15, IL-18, IL-21 or flt3, GM-CSF, G-CSF, M-CSF, IFN-α, -β, -γ, TNF-α, -β, TGF-α, -β, NGF, MDA-7 (Melanoma differentiation associated gene-7, mda-7/interleukin-24; IL-24), and the like. Further examples include, proapoptotic genes such as Fas, Bax, Caspase, TRAIL, Fas ligands, nitric oxide synthase (NOS) and the like; fusion genes which can lead to cell fusion or facilitate cell fusion such as V22, VSV and the like; tumor suppressor gene such as p53, RB, p16, p17, W9 and the like; genes associated with the cell cycle and genes which encode anti-angiogenic proteins such as endostatin, angiostatin and the like.

Other opportunities for specific genetic modification include T cells, such as tumor infiltrating lymphocytes (TILs), where the TILs may be modified to enhance expansion, enhance cytotoxicity, reduce response to proliferation inhibitors, enhance expression of lymphokines, etc. One may also wish to enhance target cell vulnerability by providing for expression of specific surface membrane proteins, e.g., B7, SV40 T antigen mutants, etc.

Although any gene or coding sequence of relevance can be used in the practice of the invention, certain genes, or fragments thereof, are particularly suitable. For example, coding regions encoding immunogenic polypeptides, toxins, immunotoxins and cytokines are useful in the practice of the invention. These coding regions include those hereinabove and additional coding regions include those that encode the following: proteins that stimulate interactions with immune cells such as B7, CD28, MHC class I, MHC class II, TAPs, tumor-associated antigens such as immunogenic sequences from MART-1, gp 100(pmel-17), tyrosinase, tyrosinase-related protein 1, tyrosinase-related protein 2, melanocyte-stimulating hormone receptor, MAGEI, MAGE2, MAGE3, MAGE12, BAGE, GAGE, NY-ESO-1, β-catenin, MUM-1, CDK-4, caspase 8, KIA 0205, HLA-A2R1701, α-fetoprotein, telomerase catalytic protein, G-250, MUC-1, carcinoembryonic protein, p53, Her2/neu, triosephosphate isomerase, CDC-27, LDLR-FUT, telomerase reverse transcriptase, PSMA, cDNAs of antibodies that block inhibitory signals (CTLA4 blockade), chemokines (MIPIα, MIP3α, CCR7 ligand, and calreticulin), anti-angiogenic genes include, but are not limited to, genes that encode METH-1, METH-2, TrpRS fragments, proliferin-related protein, prolactin fragment, PEDF, vasostatin, various fragments of extracellular matrix proteins and growth factor/cytokine inhibitors, various fragments of extracellular matrix proteins which include, but are not limited to, angiostatin, endostatin, kininostatin, fibrinogen-E fragment, thrombospondin, tumstatin, canstatin, restin, growth factor/cytokine inhibitors which include, but are not limited to, VEGFNEGFR antagonist, sFlt-I, sFlk, sNRPI, murine Flt3 ligand (mFLT3L), angiopoietin/tie antagonist, sTie-2, chemokines (IP-I0, PF-4, Gro-beta, IFN-gamma (Mig), IFNα, FGF/FGFR antagonist (sFGFR), Ephrin/Eph antagonist (sEphB4 and sephrinB2), PDGF, TGFβ and IGF-β. Genes suitable for use in the practice of the invention can encode enzymes (such as, for example, urease, renin, thrombin, metalloproteases, nitric oxide synthase, superoxide dismutase, catalase and others known to those of skill in the art), enzyme inhibitors (such as, for example, alpha1-antitrypsin, antithrombin III, cellular or viral protease inhibitors, plasminogen activator inhibitor-1, tissue inhibitor of metalloproteases, etc.), the cystic fibrosis transmembrane conductance regulator (CFTR) protein, insulin, dystrophin, or a Major Histocompatibility Complex (MHC) antigen of class I or II. Also useful are genes encoding polypeptides that can modulate/regulate expression of corresponding genes, polypeptides capable of inhibiting a bacterial, parasitic or viral infection or its development (for example, antigenic polypeptides, antigenic epitopes, and transdominant protein variants inhibiting the action of a native protein by competition), apoptosis inducers or inhibitors (for example, Bax, Bc12, Bc1X and others known to those of skill in the art), cytostatic agents (e.g., p21, p16, Rb, etc.), apolipoproteins (e.g., ApoAI, ApoAIV, ApoE, etc.), oxygen radical scavengers, polypeptides having an anti-tumor effect, antibodies, toxins, immunotoxins, markers (e.g., beta-galactosidase, luciferase, etc.) or any other genes of interest that are recognized in the art as being useful for treatment or prevention of a clinical condition. Further transgenes include those coding for a polypeptide which inhibits cellular division or signal transduction, a tumor suppressor protein (such as, for example, p53, Rb, p73), a polypeptide which activates the host immune system, a tumor-associated antigen (e.g., MUC-1, BRCA-1, an HPV early or late antigen such as E6, E7, L1, L2, etc), optionally in combination with a cytokine.

The invention further comprises combinations of two or more transgenes with synergistic, complementary and/or nonoverlapping toxicities and methods of action. In summary, the present invention provides methods for inserting transgene coding regions in specific regions of the viral vector genome.

The oncolytic adenoviral vectors of the invention comprise a heterologous coding sequence that encodes a transgene, e.g., a cytokine such as granulocyte macrophage colony stimulating factor (GM-CSF). GM-CSF is a multi-functional glycoprotein produced by T cells, macrophages, fibroblasts and endothelial cells. It stimulates the production of granulocytes (neutrophils, eosinophils & basophils) and cells of the monocytic lineage, including monocytes, macrophages and dendritic cells (reviewed in Armitage J O et al. Blood 1998 Dec. 15; 92(12):4491-508). In addition, it activates the effector functions of these cells and also appears to stimulate the differentiation of B cells.

GM-CSF has been shown to augment the antigen presentation capability of the subclass of dendritic cells (DC) capable of stimulating robust anti-tumor responses (Gasson et al. Blood 1991 Mar. 15; 77(6):1131-45; Mach et al. Cancer Res. 2000 Jun. 15; 60(12):3239-46; reviewed in Mach and Dranoff, Curr Opin Immunol. 2000 October; 12(5):571-5).

The heterologous coding sequence (transgene) is provided in operable linkage to a suitable promoter. Suitable promoters that may be employed include, but are not limited to, adenoviral promoters, such as the adenoviral major late promoter and/or the E3 promoter; or heterologous promoters, such as the cytomegalovirus (CMV) promoter; the Rous Sarcoma Virus (RSV) promoter; inducible promoters, such as the MMT promoter, the metallothionein promoter; heat shock promoters; the albumin promoter; the ApoAI promoter; and a tissue-selective promoter such as those disclosed in PCT/EP98/07380 (WO 99/25860). The invention may further comprise a second heterologous coding sequence. In one embodiment, the product of the first and second heterologous coding sequences are synergistic, having complementary functions and/or nonoverlapping toxicities and methods of action.

### Targeting of Adenvorial Vectors to Cancer Cells

In another embodiment, the adenoviral particles of the invention further comprise a targeting ligand included in a capsid protein of the particle. In one embodiment, the capsid protein is a fiber protein and the ligand is in the HI loop of the fiber protein. The adenoviral vector particle may also include other mutations to the fiber protein. Examples of these mutations include, but are not limited to those described in U.S. application Ser. No. 10/403,337, WO 98/07877, WO 01/92299, and U.S. Pat. Nos. 5,962,311, 6,153,435, 6,455,314 and Wu et al. (Flexibility of the Adenovirus Fiber is Required for Efficient Receptor Interaction. J. Virol. 2003 Jul. 1; 77(13):7225-7235). These include, but are not limited to mutations that decrease binding of the viral vector particle to a particular cell type or more than one cell type, enhance the binding of the viral vector particle to a particular cell type or more than one cell type and/or reduce the immune response to the adenoviral vector particle in an animal. In addition, the adenoviral vector particles of the present invention may also contain mutations to other viral capsid proteins. Examples of these mutations include, but are not limited to those described in U.S. Pat. Nos. 5,731,190, 6,127,525, and 5,922,315. Other mutated adenoviruses are described in U.S. Pat. Nos. 6,057,155, 5,543,328 and 5,756,086.

Accordingly, in another aspect there is provided a method of selectively killing a neoplastic cell in a cell population that comprises contacting an effective amount of the viral vectors and/or viral particles of the invention with said cell population under conditions where the viral vectors and/or particles can transduce the neoplastic cells in the cell population, replicate, and kill the neoplastic cells.

The invention further comprises adenoviral vector particles, which comprise the viral vectors of the invention. In one embodiment, the viral particles further comprise a targeting ligand included in a capsid protein of the particle. In a further embodiment, the capsid protein is a fiber protein and the ligand is in the HI loop of the fiber protein.

The adenoviral vectors of the invention are made by standard techniques known to those skilled in the art. The vectors are transferred into packaging cells by techniques known to those skilled in the art. Packaging cells provide complementing functions to the adenovirus genomes that are to be packaged into the adenovirus particle. The production of such particles requires that the vector be replicated and that those proteins necessary for assembling an infectious virus be produced. The packaging cells are cultured under conditions that permit the production of the desired viral vector particle. The particles are recovered by standard techniques. Examples of packaging cells include, but are not limited to, packaging cells that have been designed to limit homologous recombination that could lead to wild-type adenoviral particles and cells disclosed in U.S. Pat. Nos. 5,994,128, issued Nov. 30, 1999 to Fallaux, et al., and 6,033,908, issued Mar. 7, 2000 to Bout, et al. Also, viral vector particles of the invention may be, for example, produced in PerC6 or Hela-S3 cells (e.g. see U.S. patent application 60/463,143).

The viral vectors of the invention are useful in studying methods of killing neoplastic cells in vitro or in animal models. In one embodiment, the cells are mammalian cells. In a further embodiment, the mammalian cells are primate cells. In a further embodiment, the primate cells are human cells.

In one embodiment of the invention, the recombinant viral vectors and particles of the present invention selectively replicate in and lyse Rb-pathway defective cells. In the majority of tumor types, the Rb/cell cycle regulatory pathway is disrupted, suggesting that Rb-pathway disregulation may be obligatory for tumorgenesis (Strauss M, Lukass J and Bartek J. Unrestricted cell cycling and cancer. Nat Med 1995; 12:1245-1246). Rb itself is mutated in some tumor types, and in other tumor types factors upstream of Rb are deregulated (Weinberg, R A. The retinoblastoma protein and cell cycle control. Cell 1995; 81:323-330). One effect of these Rb-pathway changes in tumors is the loss of pRB binding to E2F, and an apparent increase in free E2F in tumor cells. The abundance of free E2F in turn results in high level expression of E2F responsive genes in tumor cells, including the E2F-1 gene. Accordingly, the term “Rb-pathway defective cells” may be functionally defined as cells which display an abundance of “free” E2F, as measured by gel mobility shift assay or by chromatin immunoprecipitation (Takahashi Y, Rayman J B, Dynlacht B D. Analysis of promoter binding by the E2F and pRB families in vivo: distinct E2F proteins mediate activation and repression. Genes Dev. 2000 Apr. 1; 14(7):804-16).

Cells which have mutations in genes encoding factors that phosphorylate pRB may be Rb-pathway defective cells within the meaning of the invention. pRB is temporally regulated by phosphorylation during the cell cycle. Among the factors that phosphorylate pRB is the complex of cyclin-dependent-kinase 4 (CDK4) and its regulatory subunit, D-type cyclins (CycD). CDK4 is in turn regulated by the p16 small molecular weight CDK inhibitor. Phosphorylation by CDKs reversibly inactivates pRB, resulting in transcriptional activation by E2F-DP-1 dimers and entry into S phase of the cell cycle. Dephosphorylation of pRB after mitosis causes re-entry into G1 phase. In tumor cells, any one or several of the cell cycle checkpoint proteins may be modified, leading to cell cycle deregulation and unrestricted cell cycling. Loss of the pRB-E2F-DP-1 interaction, or abundance of “free E2F,” results in derepression/activation of promoters having E2F sites. Although the inventors do not wish to be limited by these theoretical considerations, we believe that derepression of the E2F-1 promoter in the viral vectors (e.g. Ar20-1007 vector) leads to transcription of E1A, viral replication, and oncolysis.

It will be understood that the invention contemplates that any one transgene may be combined with essentially any other transgene in the tumor-selective oncolytic adenoviruses of the invention, provided that the combination is useful in the treatment of cancer. With respect to combination therapy using the tumor-selective oncolytic adenoviruses of the invention together with conventional methods of cancer therapy such as radiation or chemotherapy, the choice of chemotherapeutic agent is dependent upon the indication.

In one aspect, the present invention provides tumor-selective oncolytic adenoviruses armed with the capability of a transgene such as granulocyte-macrophage colony stimulating factor (GM-CSF), exemplified herein by Ar20-1004, Ar20-1006, Ar20-1007 and Ar20-1010.

Exemplary embodiments include the administration of Ar20-1004, Ar20-1006, Ar20-1007 and Ar20-1010 in combination with gemciabine, cisplatin, taxotere/taxol or M-VAC (combination of methotrexate, doxorubicin, and cisplatin) for treatment of bladder cancer; and Ar20-1004, Ar20-1006, Ar20-1007 and Ar20-1010 cisplatin plus 5FU, taxotere/taxol, taxol plus cisplatin or taxol plus carboplatin for treatment of head and neck cancer.

### Therapeutic Methods and Compositions of the Invention

In a further aspect of the invention, a pharmaceutical composition comprising the recombinant viral vectors and/or particles of the invention and a pharmaceutically acceptable carrier is provided. Such compositions, which can comprise an effective amount of adenoviral vectors and/or particles of this invention in a pharmaceutically acceptable carrier, are suitable for local or systemic administration to individuals in unit dosage forms, sterile parenteral solutions or suspensions, sterile non-parenteral solutions or oral solutions or suspensions, oil in water or water in oil emulsions and the like. Formulations for parenteral and non-parenteral drug delivery are known in the art. Compositions also include lyophilized and/or reconstituted forms of the adenoviral vectors and particles of the invention. Acceptable pharmaceutical carriers are, for example, saline solution, protamine sulfate (Elkins-Sinn, Inc., Cherry Hill, N.J.), water, aqueous buffers, such as phosphate buffers and Tris buffers, or Polybrene (Sigma Chemicel, St. Louis Mo.) and phosphate-buffered saline and sucrose. The selection of a suitable pharmaceutical carrier is deemed to be apparent to those skilled in the art from the teachings contained herein. These solutions are sterile and generally free of particulate matter other than the desired adenoviral virions. The compositions may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride, sodium lactate, etc. Excipients that enhance infection of cells by adenovirus may be included.

The viral vectors are administered to a host in an amount that is effective to inhibit, prevent, or destroy the growth of the tumor cells through replication of the viral vectors in the tumor cells. Such administration may be by systemic administration as herein described, or by direct injection of the vectors in the tumor. In general, the vectors are administered systemically in an amount of at least 5×10⁹ particles per kilogram body weight and in general, such an amount does not exceed 1×10¹³ particles per kilogram body weight. The vectors are administered intratumorally in an amount of at least 2×100′ particles and in general such an amount does not exceed 1×10¹³ particles. In yet another approach, the vectors are instilled into the bladder of the subject. In such cases, the bladder may be pre-treated with a bladder enhancer such as described in U.S. Ser. No. 10/327,869. The exact dosage to be administered is dependent upon a variety of factors including the age, weight, and sex of the patient, and the size and severity of the tumor being treated. The viruses may be administered one or more times. Single or multiple administrations of the compositions can be carried out with dose levels and pattern being selected by the treating physician. If necessary, the immune response may be diminished by employing a variety of immunosuppressants, or by removal of preexisting antibodies, so as to permit repetitive administration and/or enhance replication by reducing the immune response to the viruses. Administration of the adenoviral vectors of the present invention may be combined with other antineoplastic protocols, numerous examples of which are known in the art. Such antineoplastic protocols will vary dependent upon the type of cancer under treatment and are generally know to those of skill in the art.

Delivery can be achieved in a variety of ways, employing liposomes, direct injection, catheters, topical applications, inhalation, etc.

It follows that the invention provides a method of treating a subject having a neoplastic condition, comprising administering a therapeutically effective amount of an adenoviral vector of the invention to the subject, typically a patient with cancer. While the mechanism is not part of the invention, the viral vectors described herein are believed to distribute selective to tumor cells and essentially throughout a tumor mass due to the capacity for selective replication in the tumor tissue.

All neoplastic conditions are potentially amenable to treatment with the methods of the invention. Tumor types include, but are not limited to hematopoietic, pancreatic, neurologic, hepatic, gastrointestinal tract, endocrine, biliary tract, sinopulmonary, head and neck, soft tissue sarcoma and carcinoma, dermatologic, reproductive tract, respiratory, and the like. In one embodiment, the tumors for treatment are those with a high mitotic index relative to normal tissue. Exemplary types of neoplasms (cancers) that may be treated using the compositions and methods of the invention include any and all cancers which include cancer cells in which the replication competent vectors of the invention selectively replicate. Exemplary cancer types include, but are not limited to bladder cancer, breast cancer, colon cancer, kidney cancer, liver cancer, lung cancer (e.g. non-small cell lung carcinoma), ovarian cancer, cervical cancer, pancreatic cancer, rectal cancer, prostate cancer, stomach cancer, epidermal cancer, head and neck cancer, hematopoietic cancers of lymphoid or myeloid lineage, cancers of mesenchymal origin such as a fibrosarcoma or rhabdomyosarcoma, nasopharyngeal carcinoma (NPC), and other tumor types such as any solid tumor, melanoma, teratocarcinoma, neuroblastoma, glioma and adenocarcinoma.

The target cell may be of any cell or tissue type. In a preferred approach the target cell is tumor cell, typically a primary tumor cell. In one embodiment, the primary tumor cell is a cell selected from the group consisting of a lung tumor cell (e.g. a non-small cell lung tumor cell), a prostate tumor cell, a head and neck tumor cell, a bladder tumor cell, a melanoma tumor cell, a lymphoma cell and a kidney tumor cell.

Typically, the host organism is a human patient. For human patients, if a heterologous coding sequence is included in the vector, the heterologous coding sequence may be of human origin although genes of closely related species that exhibit high homology and biologically identical or equivalent function in humans may be used if the product of the heterologous coding sequence does not produce/cause an adverse immune reaction in the recipient. In one embodiment, the heterologous coding sequence codes for a therapeutic protein or therapeutic RNA. A therapeutic active amount of a nucleic acid sequence or a therapeutic gene is an amount effective at dosages and for a period of time necessary to achieve the desired result. This amount may vary according to various factors including but not limited to sex, age, weight of a subject, and the like.

The invention also provides for screening candidate drugs to identify agents useful for modulating the expression of E2F or TERT, and hence useful for treating cancer. Appropriate host cells are those in which the regulatory region of E2F or TERT is capable of functioning. In one embodiment, the regulatory region of E2F or TERT is used to make a variety of expression vectors to express a marker that can then be used in screening assays. In one embodiment, the marker is E1a and/or viral replication, both of which can be measured using techniques well known to those skilled in the art. The expression vectors may be either self-replicating extrachromosomal vectors or vectors that integrate into a host genome. Generally, these expression vectors include a transcriptional and translational regulatory nucleic acid sequence of E2F or TERT operatively linked to a nucleic acid encoding a marker. The marker may be any protein that can be readily detected. It may be a detected on the basis of light emission, such as luciferase, color, such as β-galactosidase, enzyme activity, such as alkaline phosphatase or antibody reaction, such as a protein for which an antibody exists. In addition, the marker system may be a viral vector or particle of the present invention.

In one embodiment, the viral vector or particle is used to assess the modulation of the E2F or TERT promoter. According to this embodiment, an effective amount of the viral vectors or viral particles of the invention is contacted with said cell population under conditions where the viral vectors or particles can transduce the neoplastic cells in the cell population, replicate, and kill the neoplastic cells. The candidate agent is either present in the culture medium for the test sample or absent for the control sample. The LD₅₀ of the viral vectors or particles in the presence and absence of the candidate agent is compared to identify the candidate agents that modulate the expression of the E2F or TERT gene. If the level of expression is different as compared to similar viral vector controls lacking the E2F or TERT promoter, the candidate agent is capable of modulating the expression of E2F. or TERT and is a candidate for treating cancers and for further development of active agents on the basis of the candidate agent so identified.

In a second embodiment, the candidate agent is added to host cells containing the expression vector and the level of expression of the marker is compared with a control. If the level of expression is different, the candidate agent is capable of modulating the expression of E2F and is a candidate for treating cancers involving this gene and for further development of active agents on the basis of the candidate agent so identified.

Active agents so identified may also be used in combination treatments, for example with oncolytic adenoviruses of the invention and/or chemotherapeutics.

The terms “candidate bioactive agent,” “drug candidate” “compound” or grammatical equivalents as used herein describes any molecule, e.g., protein, oligopeptide, small organic molecule, polysaccharide, polynucleotide, etc., to be tested for bioactive agents that are capable of directly or indirectly altering the cancer phenotype or the expression of a cancer sequence, including both nucleic acid sequences and protein sequences. In preferred embodiments, the bioactive agents modulate the expression profiles, or expression profile nucleic acids or proteins provided herein. In a particularly preferred embodiment, the candidate agent suppresses a cancer phenotype, for example to a normal tissue fingerprint. For example, the candidate agent suppresses a severe cancer phenotype. Generally a plurality of assay mixtures is run in parallel with different agent concentrations to obtain a differential response to the various concentrations. Typically, one of these concentrations serves as a negative control, i.e., at zero concentration or below the level of detection.

Candidate agents encompass numerous chemical classes, though typically they are organic molecules, e.g. small organic compounds having a molecular weight of more than 100 and less than about 2,500 daltons. Preferred small molecules are less than 2000, or less than 1500 or less than 1000 or less than 500 daltons. Candidate agents comprise functional groups necessary for structural interaction with proteins, particularly hydrogen bonding, and typically include at least an amine, carbonyl, hydroxyl or carboxyl group, e.g. at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992, Current Protocols in Molecular Biology (John Wiley & Sons, including periodic updates); Glover, 1985, DNA Cloning (IRL Press, Oxford); Anand, 1992, Techniques for the Analysis of Complex Genomes, Academic Press, New York; Guthrie and Fink, 1991, Guide to Yeast Genetics and Molecular Biology, Academic Press, New York; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986).

### EXAMPLES

The present invention is described by reference to the following Examples, which are offered by way of illustration and are not intended to limit the invention in any manner. Standard techniques well known in the art or the techniques specifically described below are utilized.

### Example 1

### Construction of Ar20-1007 and Ar20-1004

Plasmid pDR1F was derived from the ligation of StuI/MfeI fragments of pDr1FRgd (9731 bp) and pDr2F (867 bp). Plasmid pDr1FRgd was the product of the ligation of a 10132 bp AvrII,ClaI fragment of p5FIxHRFRGDL (Hay et al., Enhanced Gene Transfer to Rabbit Jugular Veins by an Adenovirus Containing a Cyclic RGD Motif in the HI Loop of the Fiber Knob, J Vasc Res 38:315-323 2001) with a 495 bp/AvrII,ClaI fragment of a 595 bp PCR product of p5FIxHRFRGDL that introduced a SwaI site to the vector. Plasmid pDR1F was used in the generation of adenoviral right end donor plasmids.

Adenovirus right donor plasmids were constructed for Ar20-1007 (carrying human GM-CSF cDNA) and Ar20-1004 (carrying mouse GM-CSF cDNA) viral vectors. Donor plasmid pDr20hGmF carrying the human GM-CSF cDNA with the left end ψ and the E2F-1 promoter was generated from recombination between plasmids pDR1F and pAr15pAE2fhGmF (described in WO 02/067861). Similarly, plasmid pDr20mGmF carrying the mouse GM-CSF cDNA was generated from recombination between pDR1F and pAr15pAE2fmGmF (also described in WO 02/067861).

The donor plasmids pDr20hGmF and pDr20mGmF were constructed as follows:

I. The pDR1F plasmid DNA was digested with StuI/SpeI, electrophoresed in a 0.8% agarose gel and the 7561 bp fragment was recovered and purified with a GeneClean II kit (BIO101, Inc., CA). The 7561 bp fragment was used in the ligation reactions of step III.

II. Preparation of inserts: The plasmids pAr15pAE2fhGmF (containing human GM-CSF insert) and pAr15pAE2fmGmF (containing mouse GM-CSF insert) were digested with StuI/SpeI/AscI. The digests were electrophoresed in a 0.8% agarose gel and the 4834 (from pAr15pAE2fhGmF) and 4861 (from pAr15pAE2fmGmF) base pair fragments containing the human and mouse GM-CSF inserts, respectively, were isolated from the gels and purified using a GeneClean II kit. The purified DNA fragments were used as the insert DNAs in the ligation reactions of step III.

III. The pDR1F fragment and the insert DNAs were ligated and transformed into E. coli HB101 competent cells (Invitrogen, Carlsbad, Calif.) to generate donor plasmids pDR20hGmF and pDR20mGmF. Plasmid clones were screened using restriction enzyme digestion (FspI and SpeI) and plasmids demonstrating the predicted patterns were used in the generation of large plasmids. In addition, the GM-CSF cDNAs of pDr20hGmF and pDr20mGmF were sequenced. The mouse sequence matched the predicted sequence and the human sequence contained a T->C substitution that is not expected to of any functional significance.

Large plasmids pAr20pAE2fhGmF and pAr20pAE2fmGmF were generated as follows:

The donor plasmids pDr20hGmF and pDr20mGmF were digested with FspI/SpeI. The large fragments containing the hGM-CSF or mGM-CSF cDNA were recovered from agarose gels and purified using a GeneClean II kit. Fifty to 100 ng of the DNA fragments were co-transformed into E. coli BJ5183 competent cells with 100 ng of PacI/SrfI digested pAr5pAE2fF plasmid DNA. Transformed BJ5183 cells were plated onto LB agar plates containing 100 ug/ml ampicillin and allowed to grow at 37° C. overnight. Colonies were inoculated into 2 ml LB medium containing 100 μg/ml ampicillin and incubated at 30° C. for 4-5 hours at 250 rpm. Plasmid DNA was isolated from the BJ5183 cultures by alkaline lysis (Sambrook et al., 1989). Purified plasmid DNA was resuspended in 15 ul of dH₂O and 10 μl was applied to a 0.8% agarose gel containing ethidium bromide. One microliter of mini-preps that contained large plasmids (i.e., >30 kbp) were used to transform 100□I of E. coli DH5α competent cells (Invitrogen). The efficiency of homologous recombination was observed to be higher when the transformation was carried out immediately after isolation of the mini-prep. The plasmid DNAs (pAr20pAE2fhGmF and pAr20pAE2fmGmF) obtained from the second transformation were analyzed by restriction enzyme digestion (MluI, SalI, EcoRV and XhoI) and plasmids containing the predicted RE patterns were selected for production of viral vectors Ar20-1007 and Ar20-1004.

### Viral Vector Generation and Confirmation of Viral Structure

AE1-2a clone S8 cells (S8 cells) were cultured in IMEM containing 10% heat inactivated FBS. Two μg of Swa I-digested large plasmid was transfected using the LipofectAMINE-PLUS reagent system (Life Technologies, Rockville, Md.) into S8 cells and cultured at 37° C., 5% CO₂, humidified in a 6-well plate. Seven days later, each well was amplified by a second incubation in a 6-well plate, 4 days later, the wells were pooled and transferred to T150 flasks then to 8 roller bottles after 4 additional days. After 3 days incubation in the roller bottles, the viral vector was purified by CsCl gradient. Viral vector concentrations were determined by spectrophotometric analysis (Mittereder et al., Evaluation of the Concentration and Bioactivity of Adenovirus Vectors for Gene Therapy. J Virol 70 7498-7509, 1996).

To confirm the structures of Ar20-1007 and Ar20-1004 viral genomic DNAs were isolated with a Puregene DNA Isolation Kit from Gentra Systems. The viral genomic DNAs were digested with restriction enzymes (RE) EcoRV, BsrGI, NotI, and MluI, and electrophoresed on a 0.8% agarose gel. In addition, Ar20-1007 and Ar20-1004 viruses were partially sequenced over the packaging signals, E2F promoters and the GM-CSF cDNAs.

Results

Following cloning, orientation of donor plasmids was confirmed by digestion with SpeI and FspI and the integrity of large plasmids pAr20pAE2fhGmF and pAr20pAE2fmGmF was confirmed by MluI, SalI, EcoRV and XhoI digestion.

After transfection of S8 cells, the Ar20-1007 and Ar20-1004 viral vectors were isolated and amplified. To confirm the integrity of the viruses, viral genomic DNAs were isolated and digested with restriction enzymes MluI, SalI, EcoRV and XhoI. The restriction enzyme digests showed the expected pattern. The integrity of the human and mouse GM-CSF cDNA inserts was confirmed by sequencing bp 28833 to 29828 of Ar20-1007 and bp 28827 to 29656 of Ar20-1004, respectively. The integrity of the E2F promoters was confirmed by sequencing bp 427 to 900 of Ar20-1007 and bp 440 to 909 of Ar20-1004 and the integrity of the packaging signals and left ITRs was confirmed by sequencing bp 2 to 480 of Ar20-1007 and 1 to 501 of Ar20-1004.

The junctions between the E2F promoters and the E1 regions of both viruses were found to have 3 bp deletions at nucleotides 830-832 and the 3′ untranslated region of the human GM-CSF cDNA was found to contain a single bp T->C substitution (nucleotide 29515).

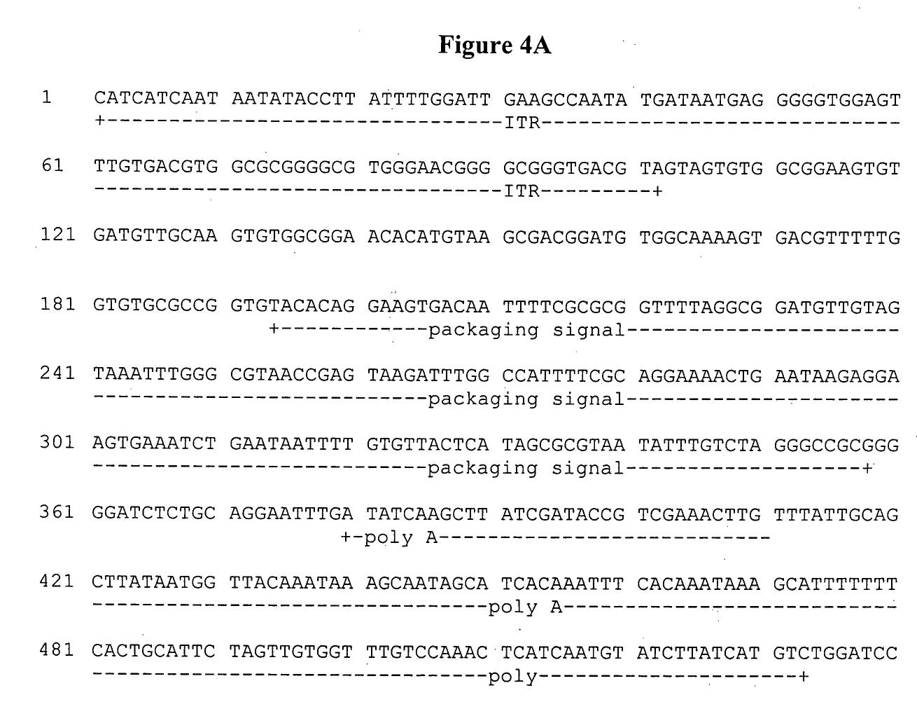

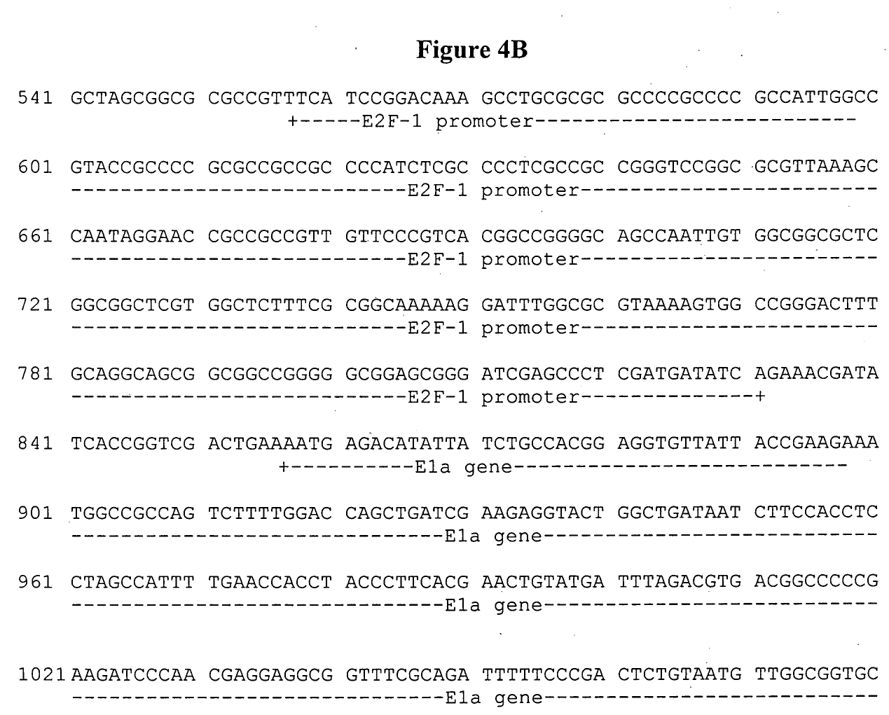

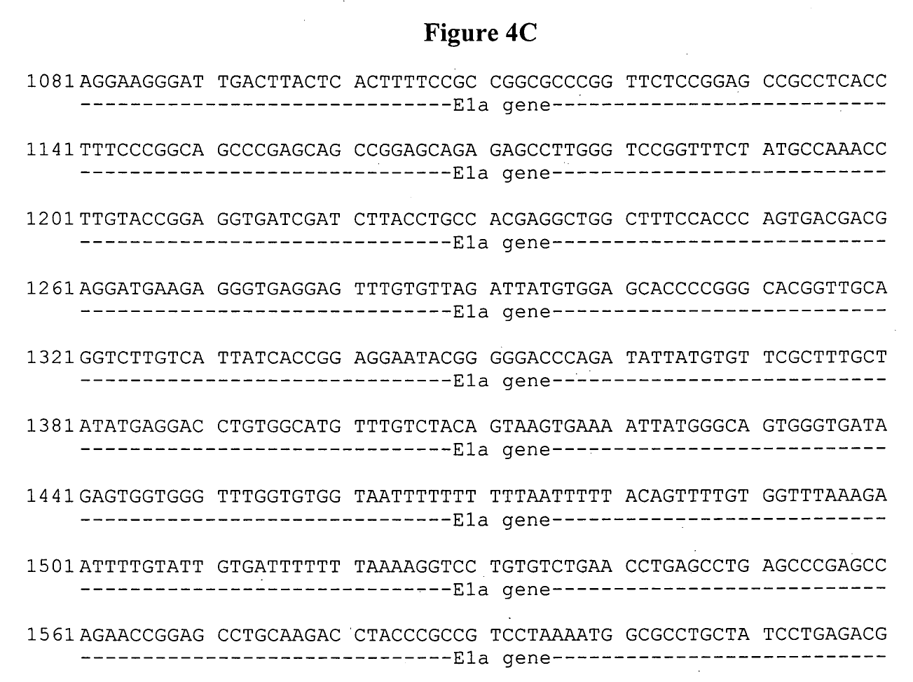

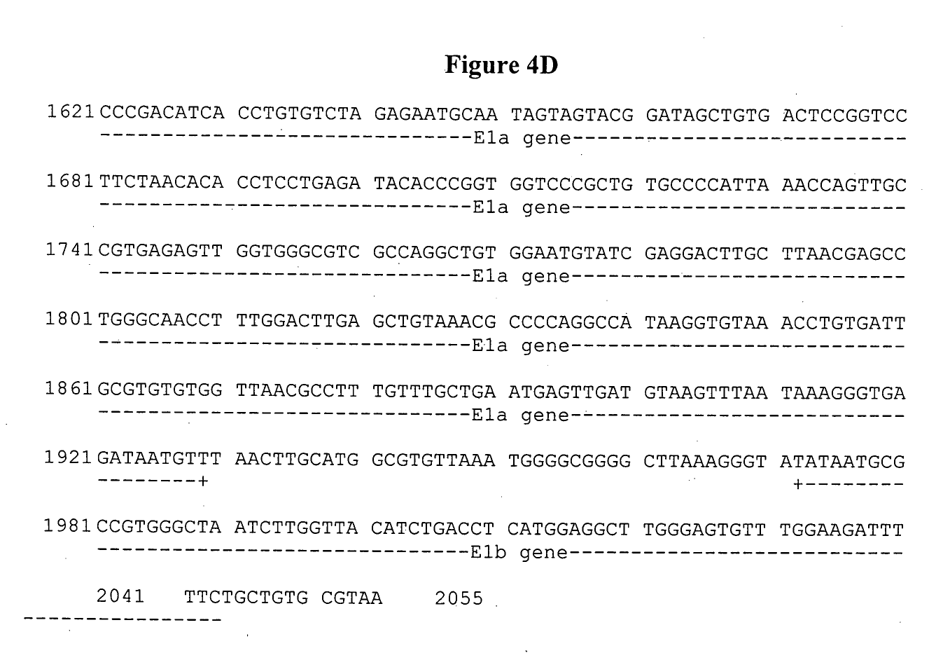

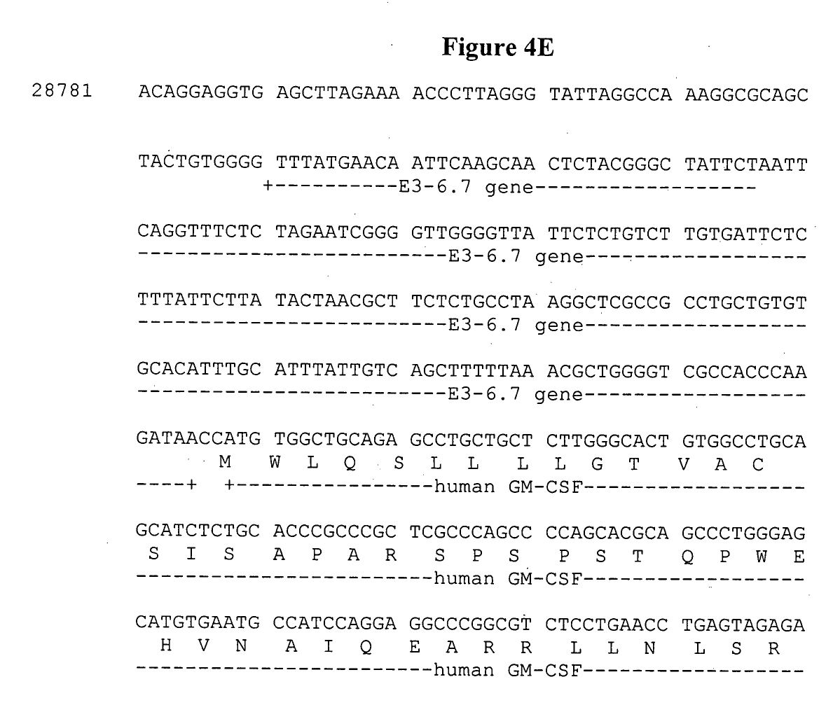

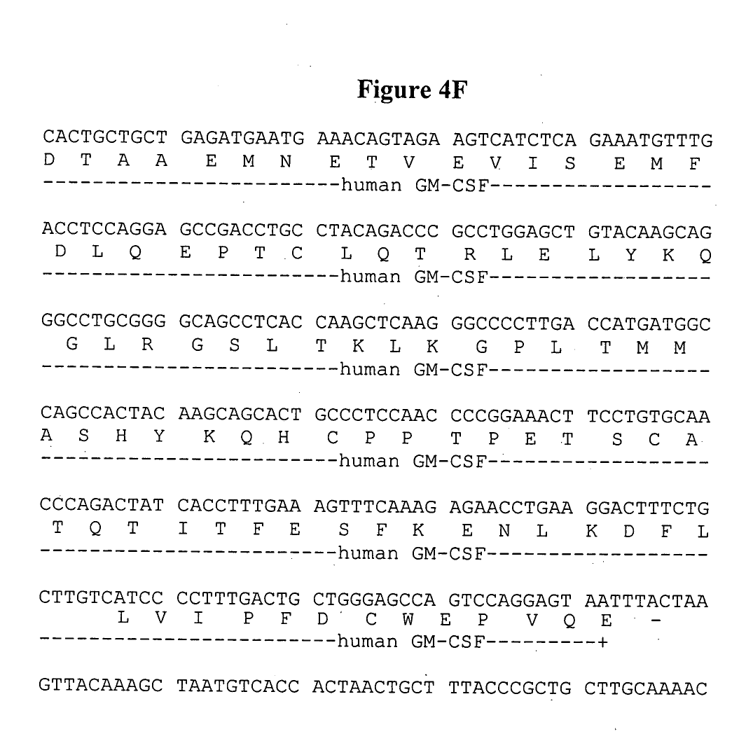

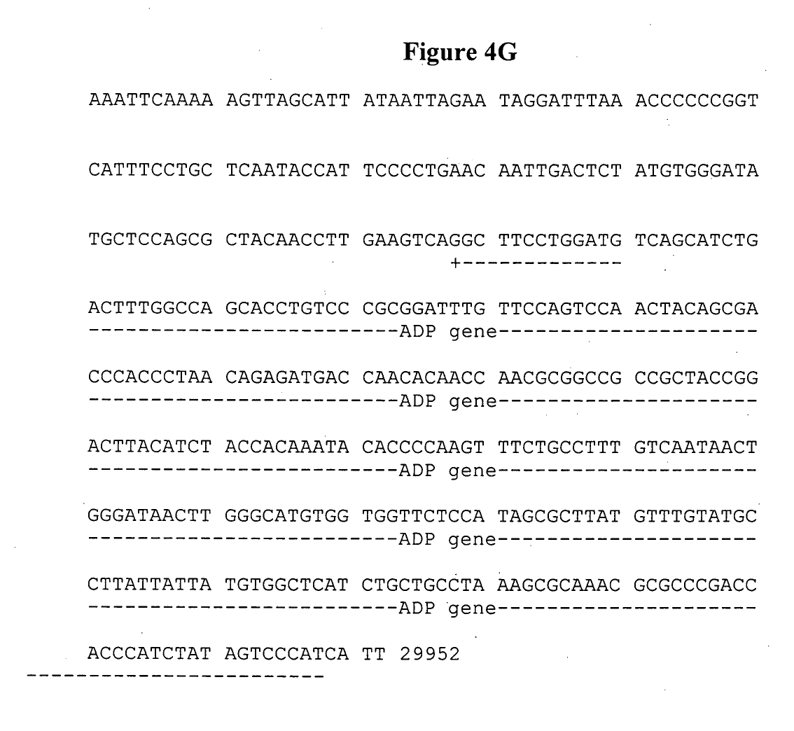

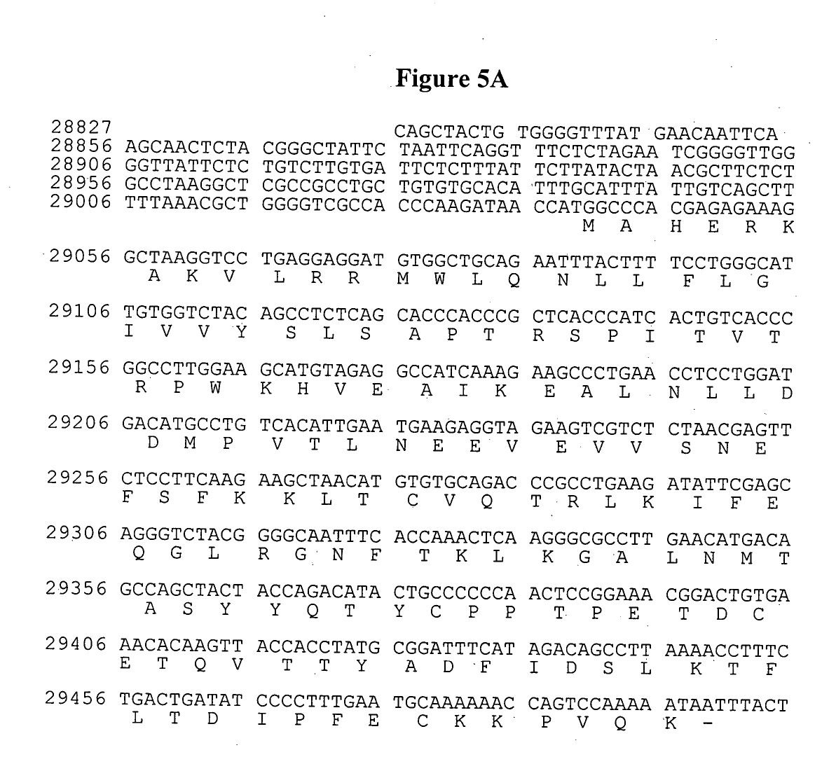

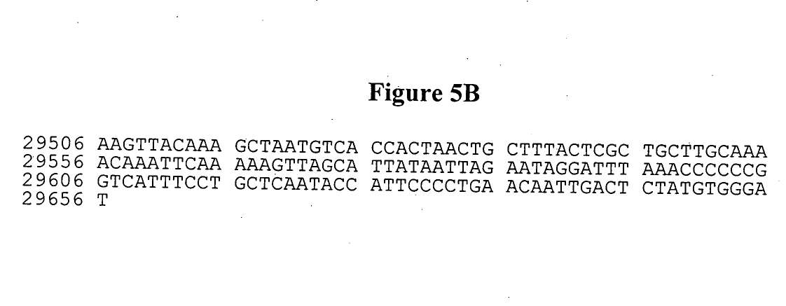

Ar20-1007 and Ar20-1004 carry human or mouse GM-CSF, respectively, in the E3-gp19 position. All the other E3 proteins, including E3-12.5, E3-6.7, E3-11.6 (ADP), E3-10.4 (RIDα), E3-14.5 (RIDβ) and E3-14.7 proteins (E3 region reviewed in (Wold et al., 1995) are retained in Ar20-1007 and Ar20-1004. Restriction digestion and partial sequencing of the viral vectors confirm the relocation of the packaging signal, integrity of the E2F promoter and the inclusion of the transgenes (FIGS. 4 and 5). There are minor deviations from the expected sequences that are not expected to have any functional effects. Base pairs 1 through 909 of Ar20-1004 have been sequenced and found have the same sequence as Ar20-1007 over the same nucleotides.

### Example 2

### Construction of Ar20-1006 and Ar20-1010

Large plasmids pAr20pATrtexhGmF and pAr20pATrtexmGmF were generated as follows:

The pDL5pATrtexF plasmid was digested with restriction enzymes AseI and BlpI, and electrophoresed in a 0.8% agarose gel to confirm the expected 9316 bp and 2140 bp DNA fragments. The digested DNA was cleaned with chloroform/phenol solution. The plasmids pAr20pAE2fhGmF and pAr20pAE2fmGmF were digested with restriction enzymes BstBI and BstZ171, and electrophoresed in a 0.8% agarose gel to confirm the expected DNA fragments. One hundred ng of AseI/BlpI digested pDL5pATrtexF (9316 bp fragment) and 100 ng of BstBI/BstZ171 digested pAr20pAE2fhGmF (32249 bp fragment) or pAr20pAE2fmGmF (32276 bp fragment) were co-transformed into BJ5186 cells. DNA minipreps from several colonies were digested with AscI. The colonies that matched the predicted RE pattern were transformed into DH5ax cells to be amplified. The final plasmids pAr20pATrtexhGmF and pAr20pATrtexmGmF were confirmed by restriction enzyme digestion with AgeI, EcoRV, NsiI and XhoI, and DNA sequencing.

Viral Vector Generation and Confirmation of Viral Structure:

AE1-2a clone S8 cells (S8 cells) were cultured in IMEM containing 10% heat inactivated FBS. Two μg of Swa I-digested large plasmid was transfected using the LipofectAMINE-PLUS reagent system (Life Technologies, Rockville, Md.) into S8 cells and cultured at 37° C., 5% CO₂, humidified in a 6-well plate. Seven days later, each well was amplified by a second incubation in a 6-well plate, 4 days later, the wells were pooled and transferred to T150 flasks then to 8 roller bottles after 4 additional days. After 3 days incubation in the roller bottles, the viral vector was purified by CsCl gradient. Viral vector concentrations were determined by spectrophotometric analysis (Mittereder et al., Evaluation of the Concentration and Bioactivity of Adenovirus Vectors for Gene Therapy. J Virol 70 7498-7509, 1996).

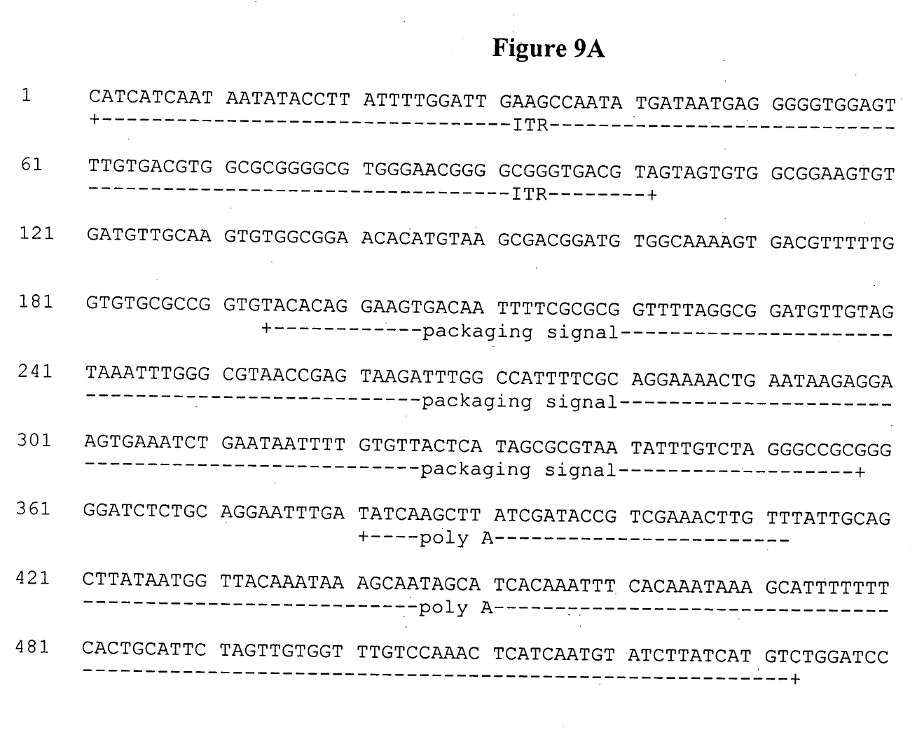

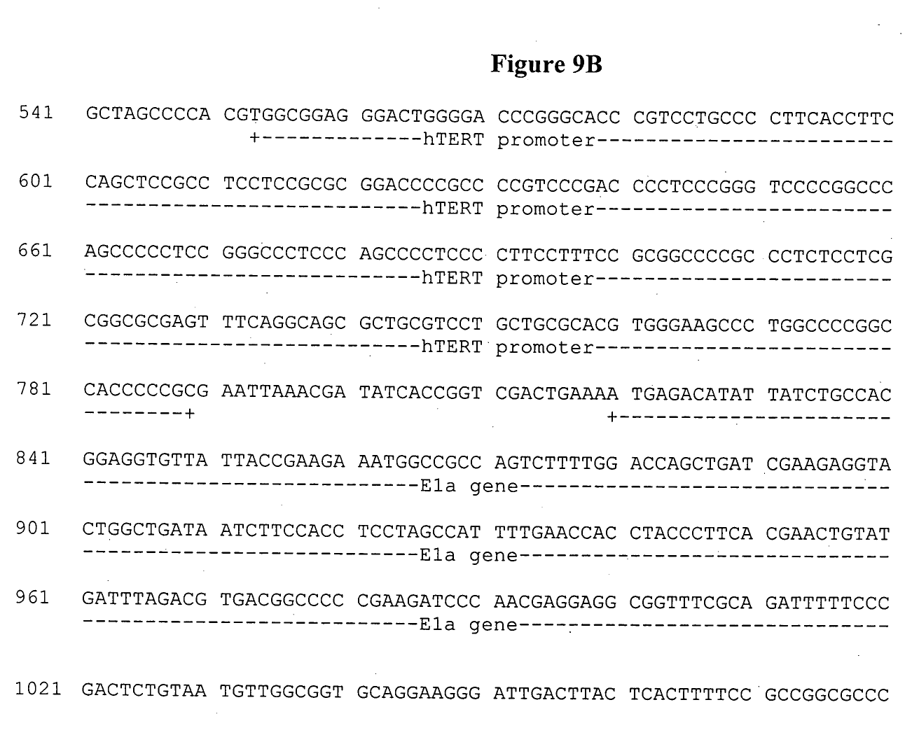

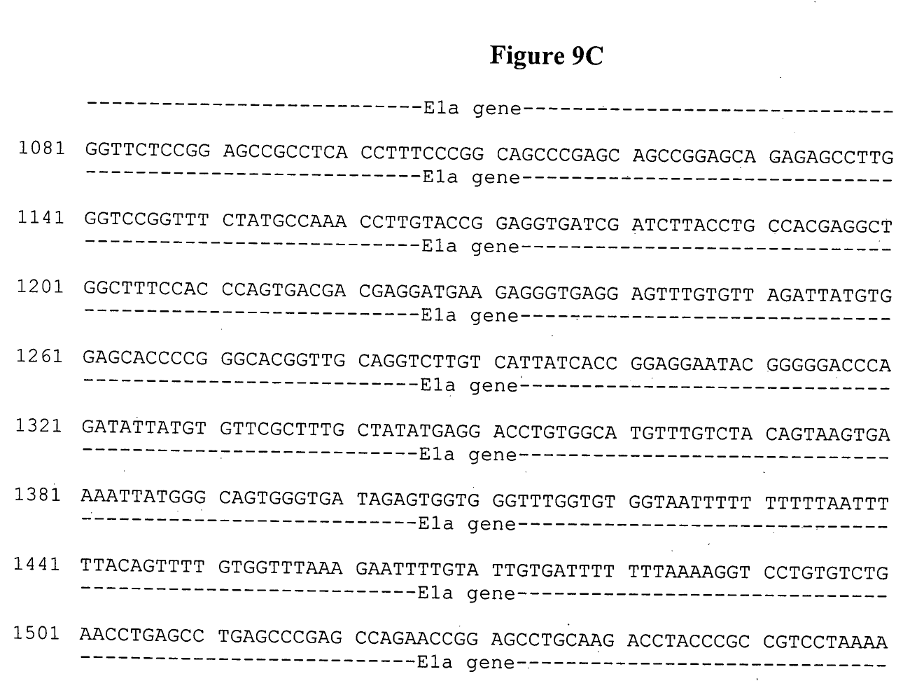

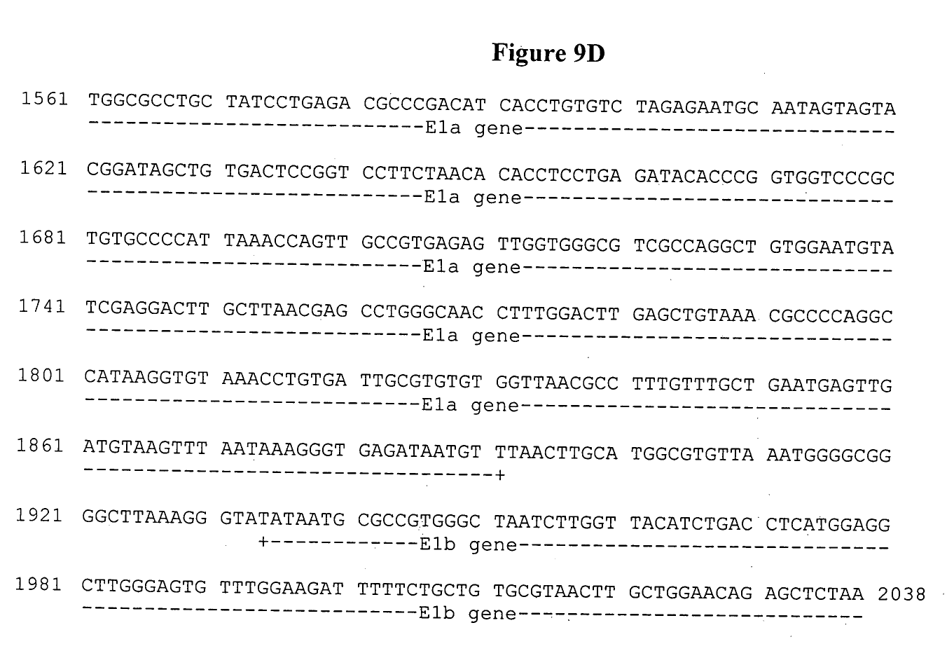

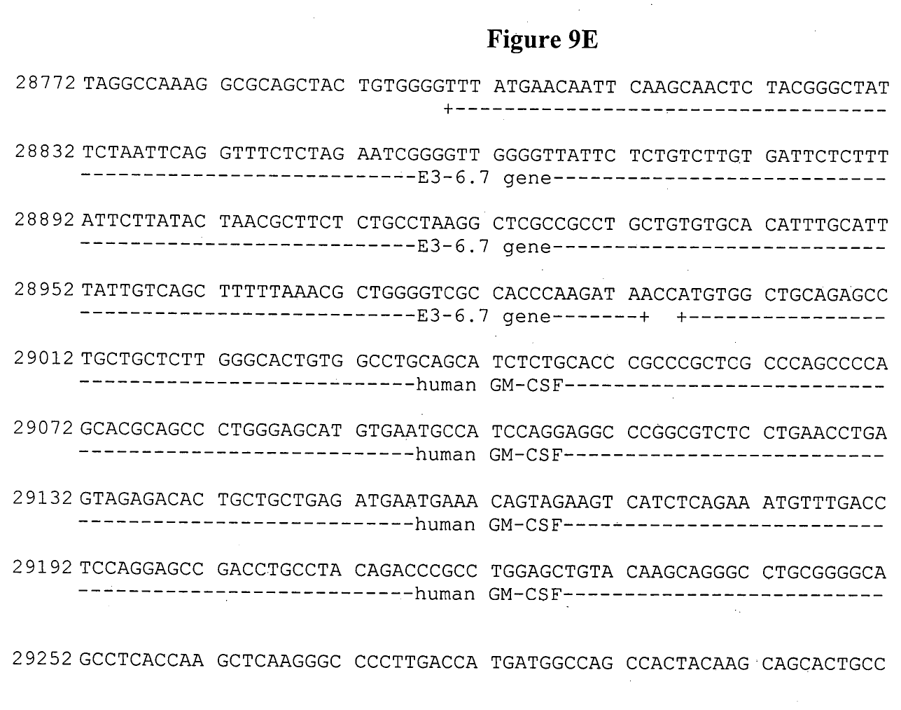

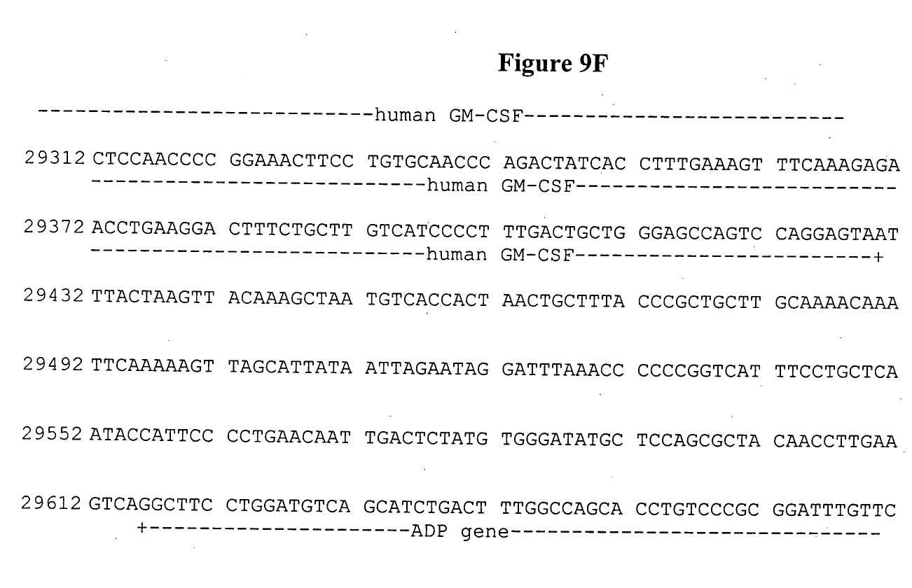

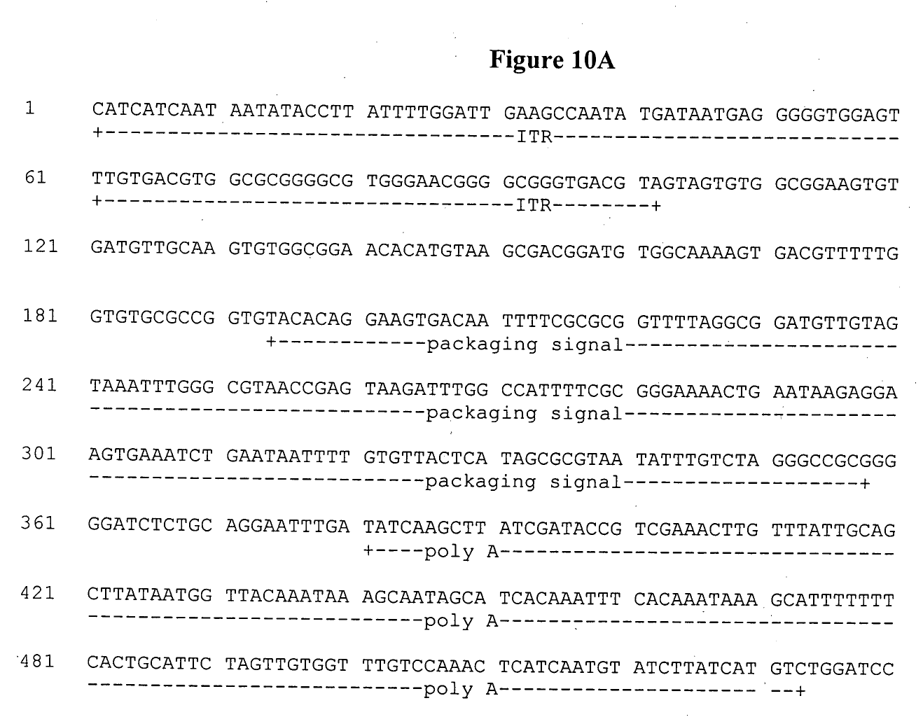

To confirm the structures of Ar20-1006 and Ar20-1010, containing the hTERT promoter and the human and mouse GM-CSF cDNAs, respectively, viral genomic DNAs were isolated with a Puregene DNA Isolation Kit from Gentra Systems. The viral genomic DNAs were digested with restriction enzymes (RE) EcoRV, BsrGI, HpaI, and EcoI, and electrophoresed on a 0.8% agarose gel. In addition, the Ar20-1006 virus was partially sequenced over the packaging signals, TERT promoters and the GM-CSF cDNA (FIG. 9).

The junctions between the E2F promoters and the E1 regions of both viruses were found to have 3 bp deletions at nucleotides 830-832 and the 3′ untranslated region of the human GM-CSF cDNA was found to contain a single bp T->C substitution (nucleotide 29515).

### Example 3

### Confirmation of E2F disregulation as target of Ar20-1007 Rationale

GM-CSF was cloned into a position under the control of the adenoviral E3 promoter. The E3 promoter is, in turn, transactivated by E1A (Horwitz M S. Adenoviruses. In: “Fields Virology, third edition,” ed Fields B N, Knipe D M, Howley P M, et al., Lippincott-Raven Publishers, Philadelphia, 1996, pp 2149-2171). Thus, ultimate control of the E3 promoter should be the result of the specificity of the E2F-1 promoter regulating the expression of the E1a gene. The Wi38-VA13 (VA13) cell line is an SV40 large T antigen (T-Ag) transformed derivative of Wi38 normal human diploid fibroblast cells. The T-Ag binds the Rb/E2F complex, resulting in the release of the E2F-1 transcription factor that is capable of activating its own promoter. As a result, VA13 cells have higher levels of E2F-1 mRNA. The location of the packaging signal ψ may impact on the selectivity of the promoter. Thus, this same cell pair was used as a model system to compare the tightness and specificity of the E3 promoters in Ar20-1007 (left and ψ and Ar15pAE2fGmF (right end ψ WO 02/067861.

Methods.

The cells were infected with Ar15pAE2fGmF or Ar20-1007 on ice for 1 hour to synchronize internalization of the viruses, and then incubated at 37° C. Quantitative PCRs for hexon DNA (as a measure of viral transduction efficiency) and E1A mRNA (as a measure of E2F-1 promoter activity) were performed after 4 or 24 hours, respectively. Also at the 24 hour timepoint, E3 promoter activation, as reflected by GM-CSF in the culture media (ELISA) was determined. To control for possible differential transduction efficiencies, E1A and human GM-CSF levels were normalized to hexon DNA levels.

TABLE A
Selective E1a gene transcription in Rb pathway disregulated cells

| | Relative copies of E1A |
|---|---|
| Group | mRNA/Adenoviral genome |
| Wi38 mock | 0 |
| Wi38 - 100ppc Ar15pAE2fhGmF | 782 ± 218 |
| Wi38 - 1000ppc Ar15pAE2fhGmF | 1338 ± 488 |
| Wi38 - 100ppc Ar20-1007 | 484 ± 400 |
| Wi38 - 1000ppc Ar20-1007 | 911 ± 83 |
| Wi38A/VA13 mock | 0 |
| Wi38A/VA13 - 100ppc Ar15pAE2fhGmF | 10593 ± 681* |
| Wi38A/VA13 - 1000ppc Ar15pAE2fhGmF | 50228 ± 13627* |
| Wi38A/VA13 - 100ppc Ar20-1007 | 31997 ± 5418* |
| Wi38A/VA13 - 1000ppc Ar20-1007 | 219478 ± 82650* |

Wi38 and Wi38-VA13 cells were infected with adenoviral vectors Ar15pAE2fGmF or Ar20-1007 at 100 and 1000 ppc for 1 hour. Real-time PCR was performed on the infected cells 24 hours post infection to determine E1A RNA levels. E1A RNA levels were normalized to hexon DNA copy number at 4 hours post-infection. *p<0.01 t-test, E1A in Wi38-VA13 vs. E1A in Wi38 infected with the same viral vector.

TABLE B
Selective GM-CSF production in Rb pathway disregulated cells

| | hGM-CSF (pg/10⁶ cells/ |
|---|---|
| Group | 24 hr/Ad genome) |
| Wi38 - 100ppc Ar15pAE2fhGmF | 52 ± 0 |
| Wi38 - 100ppc Ar20-1007 | 39 ± 10 |
| Wi38/VA13 - 100ppc Ar15pAE2fhGmF | 126822 ± 28646* |
| Wi38/VA13 - 100ppc Ar20-1007 | 83517 ± 9346* |

The supernatants from Wi38 and Wi38-VA13 cells infected with Ar15pAE2fhGMF or Ar20-1007 at 100 ppc were analyzed for hGM-CSF 24 hours following infection by ELISA. *p<0.01, t-test hGM-CSF level in Wi38-VA13 cells vs. Wi38 cells infected with the same viral vector.

Results and Conclusions.

High hGM-CSF production was observed in infected Wi38-VA13 cells and minimal production was observed in Wi38 cells. Thus, the E2F-1 promoter was selectively activated in cells with abundant E2F-1 levels, resulting in tumor cell selective production of GM-CSF. Differences in GM-CSF production between Wi38 and Wi38-VA13 cells are the sum total of a cascade of molecular events initiated by differential activation of the E2F-1 promoter, resulting in transcription/translation of E1A, initiation of viral replication and activation of the E3 promoter. The data provide strong evidence that the E2F-1 promoter in Ar20-1007 selectively regulates E1A gene transcription and downstream E3 promoter regulated GM-CSF expression in pRb-pathway defective cells. Furthermore, the data provide strong evidence that the location of the packaging signal to the left end of Ar20-1007 had no significant effect on the tumor selectivity of the promoter.

### Example 4

### Transduction of Tumor Cells In Vitro

Rationale. The ability of oncolytic adenoviruses to transduce human tumor cells is a required component of the mechanism of action. If the virus fails to enter the tumor cell, it will not be able to produce GM-CSF or replicate and lyse the cell.

Methods. Intracellular expression of the adenoviral hexon protein, as detected by flow cytometry 24 hours following infection of human H460 non-small cell carcinoma cells (NSCLC) or Hep3B (hepatocellular carcinoma) or PC3M.2AC6 (prostate carcinoma) cells was used as a measure of transduction efficiency.

TABLE C
Transduction of human tumor cells

| Cell line/ | | | | |
|---|---|---|---|---|
| viral | Percent hexon positive | | | |
| vector | mock | 10ppc | 100ppc | 1000ppc |
| H460 | 1.79 ± 0.08 | 3.46 ± 1.10 | 11.80 ± 2.36 | 45.01 ± 0.10 |
| Ar20-1004 | | | | |
| H460 | ″ | 3.00 ± 1.31 | 14.14 ± 2.59 | 50.83 ± 2.40 |
| Ar20-1007 | | | | |
| Hep3B | 1.97 ± 0.42 | 16.97 ± 0.04 | 53.54 ± 1.44 | 60.59 ± 0.13 |
| Ar20-1004 | | | | |
| Hep3B | ″ | 14.05 ± 2.62 | 53.25 ± 0.54 | 64.25 ± 1.64 |
| Ar20-1007 | | | | |
| PC3M-2Ac6 | 1.79 ± 0.88 | 2.71 ± 0.52 | 13.87 ± 0.18 | 55.17 ± 1.96 |
| Ar20-1004 | | | | |
| PC3M-2Ac6 | ″ | 1.17 ± 0.42 | 13.77 ± 0.84 | 59.93 ± 0.68 |
| Ar20-1007 | | | | |

Cells were infected with Ar20 viral vectors for 2 hours and cultured for 24 hours prior to staining with anti-hexon mAb and analyzed by FACS. Hexon expression in: A. H460 cells, B. Hep3B and C. PC3M.2AC6 cells 24 hours after being infected with the indicated viruses. Each column represents an average of two tests.

Ar20-1007 and Ar20-1004 efficiently transduced target human tumor cells in vitro (Table D). Twenty four hours after infection with Ar20-1007 or Ar20-1004, greater than 50% of the tumor cells exposed to 1000 particles per cell contained intracellular hexon. The Percent cells transduced was dose dependent.

### Example 5

### In Vitro Quantitation of Biological Activity of Virally Expressed GM-CSF

Rationale. GM-CSF production was quantitated by ELISA and bioassay in order to determine whether the GM-CSF produced following viral infection was biologically active.

Methods. GM-CSF in supernates of H460 NSCLC and PC3M.2AC6 prostate carcinoma cells infected by various particles per cell of Ar20-1007 was measured by ELISA and by ³H-thymine uptake using the GM-CSF dependent TF-1 erythroleukemia cell line.

TABLE D
In vitro production of biologically active human GM-CSF

| | | ELISA | Bioassay |
|---|---|---|---|
| | | (ng/ml/10⁶ | (ng/ml/10⁶ |
| Cell line | particles/cell | cells/24 hr) | cells/24 hr) |
| H460 cells | 1000 | 548 ± 50 | 787 ± 140 |
| | 100 | 65 ± 11 | 155 ± 84 |
| | 10 | 9 ± 1 | 22 ± 9 |
| PC3M-2AC6 cells | 1000 | 339 ± 56 | 597 ± 43 |
| | 100 | 848 ± 73 | 2677 ± 2106 |
| | 10 | 50 ± 1 | 93 ± 104 |

Duplicate wells of human H460 NSCLC tumor cells or PC3M-2Ac6 prostate carcinoma cells were infected with Ar20-1007 the indicated particles/cell ratio for 24 hours. Cell supernatants were collected and tested for total GM-CSF protein by ELISA (in duplicate), and for GM-CSF activity using a proliferation bioassay (in triplicate). Data represent the average ± standard deviation of replicate wells in the same units of ng/10⁶ cells/24 hours.

Results and conclusions. The amounts of GM-CSF detected by ELISA and by bioassay using proliferation of TF-1 cells were similar following in vitro infection of H460 and PC3M.2AC6 cells. ELISA serves as an accurate, convenient and rapid method of quantifying GM-CSF levels. These data also provide an in vitro dose response curve of GM-CSF production. GM-CSF production ranges from several hundred ng/10⁶ cells/24 hours when infected with 100 to 1000 ppc, to 10 to 100 ng/10⁶ cells/24 hours at 10 ppc. The data show that the total GM-CSF produced (as measured by ELISA) is biologically active (as measured by the bioassay). At 100 ppc, GM-CSF production in both cell lines exceeded the 40 ng/ml/10⁶ cells/24 hr level that has been shown necessary to induce potent, long-lasting antitumor immunity in ex vivo tumor vaccination models (Dranoff et al., Vaccination with irradiated tumor cells engineered to secrete murine GM-CSF stimulates potent, specific and long-lasting anti-tumor immunity. Proc National Acad Sci 90:3539-3543, 1993; Simons J W, Jaffee E M, Weber C E, et. al. (1997) Bioactivity of autologous irradiated renal cell carcinoma vaccines generated by ex vivo granulocyte-macrophage colony-stimulating factor gene transfer. Cancer Res. 57:1537-1546).

### Example 6

### Selectivity of Ar20-1007 as Measured by In Vitro Cytotoxicity Assays

Rationale. The cytotoxicity of Ar20-1007 on target human tumor cells and non-target primary human cells was compared to the cytotoxicity of Add/1520 (in-class competitor), wild type Ad5 (non-tumor selective virus) and Add/312 (E1a deleted replication defective virus).

Methods. Colorimetric MTS-based cytotoxicity assays (Bristol et al., In vitro and in vivo activities of an oncolytic adenoviral vector designed to express GM-CSF. Mol Ther 7: 755-764, 2003) were performed using Ar20-1007, Addl1520, Ad5 and Add/312 using human tumor cells: Hep3B (hepatocellular carcinoma), SW620 (colon carcinoma), LNCaP-C4-2 (prostate carcinoma) and PC3M.2AC6 (prostate carcinoma) and primary and non-transformed human cells: hAEC (aortic endothelial cells), hMEC (mammary epithelial cells), hREC (renal endothelial cells), hUVEC (umbilical vein endothelial cells), NHLF (normal lung fibroblasts), and MRC-5 (passage limited lung fibroblast cell line). The experimental EC₅₀ values of Ar20-1007 vs Ad5 and Ar20-1007 vs. Addl1520 were compared using stimulation indexes (SI) to estimate the differences in selectivity for tumor cells between the three viruses (Ar20-1007, Ad5 and Addl1520). Selectivity indices greater than 1 indicate tumor cell selectivity by a given viral vector and the greater the Si, the greater the viral vector selectivity for tumor cells over primary cells.

TABLE E
Tumor cell selectivity of GMI007

| | Primary cell type (SI of Ar20-1007 vs. wt Ad5 and SI of Ar20-1007 vs. Addl1520) | | | | | | | | | | | | | |
|---|---|---|---|---|---|---|---|---|---|---|---|---|---|---|
| Tumor line | hAEC | | hMEC | | hREC | | hUVEC | | hMVEC | | NHLF | | MRC-5 | |
| Hep3B | 43 | 307 | 1.5 | 75 | 0.68 | 68 | 113 | 15 | 15 | 20 | 0.76 | 8 | 14 | 19 |
| SW620 | 99 | 148 | 3.5 | 438 | 1.56 | 312 | 261 | 75 | 34 | 100 | 1.75 | 39 | 32 | 94 |
| LNCaP-C4-2 | 132 | 322 | 4.7 | 92 | 2.1 | 72 | 348 | 16 | 45 | 21 | 2.3 | 8 | 43 | 20 |
| PC3M-2Ac6 | 32 | 40 | 1.1 | 11 | 0.5 | 9 | 83 | 2 | 11 | 3 | 1.56 | 1 | 10 | 2 |

Selectivity index (SI) for Ar20-1007. The SI values were computed as described (Bristol et al., 2002a) for each tumor line (listed on left) compared to each primary cell type (listed across top of table). Values greater than 1 demonstrate tumor selectivity. Shown in red italics are the ratios of GMI007 to Add/1520 to demonstrate the fold increase in tumor selectivity with respect to cytotoxicity of GMI007 vs an in-class competitor. RD-2002-51231.

Results and conclusions. GMI007 is tumor selective in 25/28 comparisons vs. Ad5 and is more tumor selective than Addl1520 in 27/28 comparisons.

### Example 7

### In Vivo Spread of Ar20-1007 Through a Tumor

Rationale. Oncolytic adenoviruses are designed to selectively replicate and spread in target tumor cells. Thus, following the initial viral vector inoculation in vivo, there should be a time-related increase in virally transduced tumor cells.

Methods. Human prostate carcinoma PC3M.2AC6 cells were inoculated into the flanks of female nude mice. When the tumors reached ˜100 to 200 mm³, a single intratumoral injection of 1.54×10¹⁰ particles of Ar20-1007, negative control replication defective virus Addl312 or HBSS was administered. Tumors were measured in two dimensions then excised 2, 6 and 11 days after the injection and single cell suspensions were prepared and stained for intracellular expression of adenoviral hexon before analysis by flow cytometry. Tumor volumes were calculated using the formula V=W²Lπ/6; V, volume; W, width; L, length.

TABLE F
Spread of Ar20-1007 in PC3M.2AC6 tumors in vivo

| | % hexon positive tumor cells | | |
|---|---|---|---|
| Group | Day 2 | Day 6 | Day 11 |
| HBSS | 0.46 ± 0.08 | 0.44 ± 0.07 | 0.36 ± 0.01 |
| Addl312 | 0.54 ± 0.09 | 9.15 ± 2.25 | 2.21 ± 0.31 |
| Ar20-1007 | 2.15 ± 0.41 | 53.51 ± 4.0* | 13.07 ± 1.74* |

On days 2, 6, and 11 following a single intratumoral administration of 1.54×10¹⁰ viral particles or HBSS, tumors were analyzed for hexon staining using intracellular flow cytometry. The percentage of hexon positive cells from each mouse is displayed as the mean ±SEM (n=10). *: p<0.001 compared to HBSS and Add/312, ANOVA.

TABLE G
Effect of a single intratumoral injection of
Ar20-1007 on PC3M.2AC6 tumor volume in vivo

| | Tumor volume, mm³ | | |
|---|---|---|---|
| Group | Day 2 | Day 6 | Day 11 |
| HBSS | 207 ± 24 | 305 ± 42 | 537 ± 50 |
| Addl312 | 171 ± 22 | 295 ± 31 | 400 ± 74 |
| Ar20-1007 | 177 ± 25 | 280 ± 37 | 274 ± 56* |

On days 2, 6, and 11 following a single intratumoral administration of 1.54×10¹⁰ viral particles or HBSS, 10 mice from each group were sacrificed and tumor volumes were measured prior to processing for hexon flow cytometry. Each bar represents the average tumor volume±SEM of 10 mice. *: p<0.05 compared to HBSS, ANOVA.

Results and conclusions. The results (Table F) demonstrated significant viral spread through the tumor. On day 2 following the single dose of viral vector, only a few (2 to 3%) cells were positive for hexon. By day 6, greater than 50% of the tumor cells had been infected by virus. On day 11, the percentage of infected cells had decreased to approximately 13%. This could indicate that a single intratumoral injection is not adequate to spread to all tumor cells. In addition, in this model, tumor cell proliferation may be faster than viral spread. Nevertheless, a single injection of Ar20-1007 was sufficient to significantly delay tumor growth (Table G).

### Example 8

### Evaluation of GM-CSF Expressed in Nude Mice Bearing Subcutaneous Human PC3M-2Ac6 Prostate Tumors after Intratumoral Injection

The human prostate carcinoma cell line PC3M-2Ac6 is obtained from Dr. Peter Lassota (Novartis, Summit, N.J.) (Proc. Ann. Assoc. Cancer Res., 43:737, abstract 3652 (2002)). The PC3M-2Ac6 cells are cultured in RPMI1640, with 10% FBS. Cells are incubated at 37° C. in 5% CO₂ humidified air and subcultured twice weekly.

1. Mouse Tumor Model

Female athymic nude (nu/nu) mice are purchased from Harlan-Sprague-Dawley (Indianapolis, Ind.) and kept for one week in quarantine before initiation of the study. Mice are injected subcutaneously at 7-8 weeks of age with 3×10⁶ PC3M-2Ac6 cells in the right hind flank in a volume of 100 μL (PBS diluent), using a 27-gauge needle, 0.5 cc insulin syringe (Becton-Dickinson). Tumor growth is measured in two dimensions using an electronic caliper every other day beginning on the eighth day after injection of the cells. Mice are entered into studies after 10-14 days when tumor volumes reached 100-300 mm³ [calculated as volume=(W²xL)π/6 (O'Reilly, et al 1999)]. Mice are recaged (regrouped) to yield groups with similar average tumor volumes and intratumoral injection of adenoviral vectors is initiated. Viral vectors are diluted to the appropriate dose in HBSS to deliver a volume of 50 μL per tumor using a 27-gauge needle, 0.5 cc insulin syringe. Five injections are made intratumorally on an every other day schedule (Monday-Wednesday-Friday-Monday-Wednesday). On days of injection, the needle is inserted into the tumors at different entry points such as to distribute the viral vector throughout the tumor. Mice are monitored daily for adverse reactions to the injections. On study days 2, 7, 11, 14, and 21, five mice per group are terminally bled. Immediately afterward, mice are sacrificed and their tumor removed. Serum and tumor extracts are prepared and frozen for analysis at a later date.

2. Tumor Harvest and Preparation for GM-CSF ELISA Assay

On study days 2, 7, 11, 14, and 21 mice are sacrificed and the tumor is removed. Samples are kept frozen at −80° C. until the day of the assay.

Briefly, tumor samples are collected by resecting the whole tumor and removing the skin, then placing the tumor into lysing matrix tubes (Bio101 Co., cat.#6540-401). Tumors are weighed, and then homogenized in Reporter Lysis Buffer (Promega Corp., Madison, Wis.) at a ratio of 250 uL lysis buffer per 50 mg of tumor tissue. Large tumors (>1500 mm3) are minced using a razor blade and a smaller sample (150-250 mg) is used for the extract. Tissue disruption is performed for 30 seconds in a FastPrep 120 instrument (Bio101 Co.). Homogenates are centrifuged (14,000×g) for 30 minutes at 4° C., then the soluble tumor extract is removed to a new tube and frozen at −80° C. until the day of the assay. Protein concentration is determined by the BioRad Protein Microassay procedure (Bradford assay) in order to normalize the GM-CSF level in each tumor.

3. GM-CSF ELISA

The ELISA kits are purchased from R&D Systems (Minneapolis, Minn.) and the accompanying protocol is followed.

4. Statistical Analyses

Statistical tests are done using the SigmaStat software program (SPSS Inc.). All pairwise multiple comparison procedures (Dunn's method or the Tukey test) are performed to test for significance among the three dose levels. A p value of <0.05 is considered to be significant. The area under the curve (AUC) and Cmax analyses are performed using GraphPad Prism software. The AUC is calculated using first X=day 21.

### Example 9

### Results of Pharmacokinetic Evaluation of GM-CSF Expressed by the Ar20-1004 Oncolytic Adenovirus Following Intratumoral Injections in Nude Mice Bearing Subcutaneous Human PC3M-2Ac6 Prostrate Tumors

The pharmacokinetic analysis of murine GM-CSF expressed by the Ar20-1004 oncolytic viral vector is analyzed following five intratumoral injections of PC3M-2Ac6 tumor-bearing nude mice. Three dose groups are injected that covered a 4 log unit viral particle (vp) range (1.54×10⁶, 1.54×10⁸, and 1.54×10¹⁰ vp). GM-CSF is measured by ELISA from serum and tumor extracts recovered at several time points over the 21 day study. The AUC and Cₘₐₓ values are calculated from the ELISA results.

The results from serum-derived GM-CSF samples are shown in Table I. The data shows dose-dependent GM-CSF expression on days 2, 7, and 11. This dependency on vp dose is not evident on the later study days 14 or 21. The difference in GM-CSF level between 1.54×10⁶ vp and 1.54×10⁸ vp is significant on day 7, however, there are no other statistically different values when comparing the next higher vp dose groups.

TABLE I
GM-CSF expressed in serum after
Ar20-1004 intratumoral injection

| Dose (vp) | Study Day | pg GM-CSF/ml serum |
|---|---|---|
| HBSS | 2 | Nd |
| | 7 | 0.5 +/− 1.2 |
| | 11 | Nd |
| | 14 | Nd |
| | 21 | Nd |
| 1.54 × 106 | 2 | 15 +/− 15 |
| | 7 | 6 +/− 7 |
| | 11 | 10 +/− 8 |
| | 14 | 20 +/− 15 |
| | 21 | 1 +/− 3 |
| 1.54 × 108 | 2 | 182 +/− 67 |
| | 7 | 628 +/− 657 * |
| | 11 | 66 +/− 51 |
| | 14 | 153 +/− 188 |
| | 21 | 1 +/− 2 |
| 1.54 × 1010 | 2 | 31562 +/− 18367 * |
| | 7 | 1051 +/− 1117 * |
| | 11 | 644 +/− 1421 |
| | 14 | 16 +/− 19 |
| | 21 | 6 +/− 6 |

Murine GM-CSF expressed by Ar20-1004 in mouse serum. Nude mice bearing PC3M-2Ac6 tumors are injected on study days 1, 3, 6, 8, and 10 with HBSS or Ar20-1004 with the doses indicated. On study days 2, 7, 11, 14, and 21 mice are bled and the serum is tested by ELISA for murine GM-CSF expression. Data represent the average plus SD (n=5/group). *, indicates p<0.05 vs. 1.54×10⁸ vp dose. HBSS-treated mice do not express detectable levels of endogenous GM-CSF. On day 21, only 1 of 5 mice injected with 1.54×10⁶ or 1.54×10⁸ vp has detectable levels of GM-CSF.

Tumors injected with 1.54×10⁶ vp express GM-CSF that is relatively stable over the time course of the study. Tumors injected with 1.54×10⁸ vp express GM-CSF that is relatively stable during the first 14 days, but the amount of GM-CSF detected in the serum then decreases approximately 100-fold between day 14 and day 21. Tumors injected with 1.54×10¹⁰ vp express a copious amount of GM-CSF that peaked on day 2 but then decreases by 4 log units gradually over the time course of the study. No murine GM-CSF is detected in mouse serum following intratumoral injections of HBSS.

From these data the area under the curve (AUC) and Cₘₐₓ is calculated to estimate the total systemic GM-CSF exposure and peak GM-CSF expression in mice injected with Ar20-1004 by the intratumoral route. Table J shows a 21-fold increase in total GM-CSF exposure between the 1.54×10⁶ and 1.54×10⁸ vp dose groups, and a 20-fold increase between the 1.54×10⁸ and 1.54×10¹⁰ vp dose groups. The time to reach the Cₘₐₓ calculated from the data is inversely proportional to the vp dose, as the highest vp dose peaks on day 2 whereas the lowest vp dose peaks on day 14. This may reflect the fact that treatment of tumors with 1.54×10⁸ and 1.54×10¹⁰ vp doses of Ar20-1004 began to decrease tumor volume over the 21 day time course and therefore are producing less GM-CSF.

TABLE J
Serum GM-CSF calculations

| Dose level | Area under curve, ng/mL-min | C_{max,} ng/mL (Peak day) |
|---|---|---|
| 1.54 × 10⁶ vp | 291 | 0.02 (Day 14) |
| 1.54 × 10⁸ vp | 6,150 | 0.63 (Day 7) |
| 1.54 × 10¹⁰ vp | 124,000 | 31.6 (Day 2) |

Area under the curve and Cₘₐₓ calculations for murine GM-CSF expression by Ar20-1004. The analysis was performed using Prism software.

The results from GM-CSF expression in tumor extracts are shown in Table K. The data demonstrates dose-dependent GM-CSF expression on all study days. Similar to the serum-derived samples, there are no significant differences between next higher vp dose groups except between the 1.54×10⁸ vp and 1.54×10¹⁰ vp groups on day 11.

Tumors injected with 1.54×10⁶ vp express GM-CSF that gradually increases approximately 20-fold between day 2 and day 14 and is maintained at 167 pg/mg protein at day 21. Tumors injected with 1.54×10⁸ vp express GM-CSF that increases approximately 10-fold between day 2 and day 7, maintains approximately 2,500 pg/mg until day 14, then decreases approximately 15-fold by day 21. Tumors injected with 1.54×10¹⁰ vp express GM-CSF that peaks on day 2 at 29,400 pg/mg but remains above 2,000 pg/mg over the time course of the study.

TABLE K
GM-CSF expressed in tumor extract after
Ar20-1004 intratumoral injection

| Dose (vp) | Study Day | pg GM-CSF/ml serum |
|---|---|---|
| HBSS | 2 | 0.2 +/− 0.1 |
| | 7 | 0.1 +/− 0.1 |
| | 11 | 0.1 +/− 0.3 |
| | 14 | 0 +/− 0.1 |
| | 21 | 0.9 +/− 1.0 |
| 1.54 × 106 | 2 | 23 +/− 8 * |
| | 7 | 70 +/− 24 * |
| | 11 | 133 +/− 47 * |
| | 14 | 534 +/− 300 |
| | 21 | 168 +/− 154 |
| 1.54 × 108 | 2 | 272 +/− 175 |
| | 7 | 3536 +/− 1392 |
| | 11 | 2986 +/− 2837 * |
| | 14 | 2494 +/− 2651 |
| | 21 | 176 +/− 148 |
| 1.54 × 1010 | 2 | 29351 +/− 17458 |
| | 7 | 29171 +/− 44628 |
| | 11 | 8657 +/− 3440 |
| | 14 | 4475 +/− 2856 |
| | 21 | 1896 +/− 2845 |

Nude mice bearing PC3M-2Ac6 tumors are injected on study days 1, 3, 6, 8, and 10 with HBSS or Ar20-1004 with the doses indicated. On study days 2, 7, 11, 14, and 21 mice are sacrificed and the tumor is removed. A tumor extract is prepared and tested for murine GM-CSF expression by ELISA. Data represent the average plus SD (n=5/group). *, indicates p<0.05 vs. 1.54×10¹⁰ vp dose. All Ar20-1004 injected tumors are positive at all time points.

The total exposure to GM-CSF at the tumor is calculated and the data is shown in Table L. The tumor extract values are similar to the serum-derived values with respect to the dose-dependent GM-CSF expression patterns (6-10-fold GM-CSF expression increases with increasing vp dose) and the time to reach the peak expression level.

TABLE L
Tumor extract GM-CSF calculations

| Dose level | Area under curve, ng/mL-min | C_{max,} ng/mg (Day) |
|---|---|---|
| 1.54 × 10⁶ vp | 5,900 | 0.53 (D 14) |
| 1.54 × 10⁸ vp | 57,800 | 3.54 (D 7) |
| 1.54 × 10¹⁰ vp | 380,000 | 29.4 (D 2) |

Area under the curve and Cₘₐₓ calculations for murine GM-CSF expression by Ar20-1004. The analyses are performed using Prism software.

We report here that the level of murine GM-CSF expressed in vivo following intratumoral injection of the Ar20-1004 oncolytic viral vector is dose-dependent, as measured in mouse serum and from tumor extracts. This data is important to show as this represents one route of injection for the viral vector particles of the present invention that encode human GM-CSF. GM-CSF is expressed following injection of the high dose (1.54×10¹⁰ vp) of Ar20-1004 in tumors at high levels and for at least 11 days following five vector injections (29 ng/mg on day 2 decreasing to 1.9 ng/mg on day 21). Moreover, the Cₘₐₓ for the serum level of GM-CSF expressed by Ar20-1004 on day 2 (31.6 ng/mL) surpasses the maximal concentration observed following administration of 250 μg/m² of Sargramostim (recombinant human GM-CSF) via intravenous (5.0 to 5.4 ng/mL) or subcutaneous routes (1.5 ng/mL) in human GM-CSF pharmacokinetic studies (Schwinghammer, et al. Pharmacokinetics of recombinant human granulocyte-macrophage colony stimulating factor (GM-CSF) after intravenous and subcutaneous injection. Pharmacotherapy; 2:105 (abstract 60) 1991).

The persistent expression of GM-CSF at therapeutic levels will likely be necessary to induce a robust cell-mediated anti-tumor response, as well as a strong local inflammatory response. In light of the levels of GM-CSF expressed by Ar20-1004 at 1.54×108 and 1.54×1010 particles/injection observed here, it should be noted that these levels are 1-3 log units higher doses than efficacious doses using a Hep3B xenograft tumor model in nude mice. Therefore, if lower efficacious doses are administered intratumorally in a therapeutic study, the GM-CSF expressed is expected to be lower yet induce the anti-tumor activities observed.

It is worth noting that the levels of GM-CSF expressed by Ar20-1004 were similar to the pharmacokinetic profile a similar vector platform that expresses murine GM-CSF and contains the viral packaging signal on the right end of the virus genome (WO 02/067861). In addition, the time to reach peak GM-CSF expression is similar between the two vector platforms. Thus, the location of the virus packaging signal does not appear to impact the level or persistence of GM-CSF expression by these viral vectors.

The Ar20-1004 viral vector expresses copious amounts of murine GM-CSF following a regimen of five intratumoral injections in the PC3M-2Ac6 tumor xenograft model, which represents the one route of viral vector injections. GM-CSF is expressed at a level considered sufficient to generate a cell-mediated immune response, which is approximately 35 ng/10⁶ cells/24 hours (Dranoff et al., Vaccination with irradiated tumor cells engineered to secrete murine GM-CSF stimulates potent, specific and long-lasting anti-tumor immunity. Proc National Acad Sci 90:3539-3543, 1993; Simons J W, Jaffee E M, Weber C E, et. al. (1997) Bioactivity of autologous irradiated renal cell carcinoma vaccines generated by ex vivo granulocyte-macrophage colony-stimulating factor gene transfer. Cancer Res. 57:1537-1546). Further testing of the Ar20-1004 and Ar20-1007, which express a murine and human GM-CSF molecule, respectively, is warranted.

### Example 10

### In Vivo Efficacy in Hepatocellular Carcinoma and Prostate Cancer Xenograft Tumor Models

Rationale. The efficacy of Ar20-1007 was compared to a.) Addl312, a replication defective virus, b.) Addl1520, a virus molecularly identical to a virus that is being tested in clinical trials, and Ar20-1004. The experiments were carried out using three subcutaneous human tumor xenograft models (Hep3B hepatocellular carcinoma, and PC3M.2Ac6 and LnCaP-FGC prostate carcinoma cells) in immunodeficient nude or SCID mice. These studies provided a rigorous test of the efficacy of Ar20-1007 versus Add/1520, a virus in clinical trials. In addition, the comparison of Ar20-1007 (producing human GM-CSF, biologically inactive in a mouse) to Ar20-1004 (producing mouse GM-CSF) provided an assessment of the contributions of viral replication and biologically active GM-CSF to the overall response in immunodeficient mice.

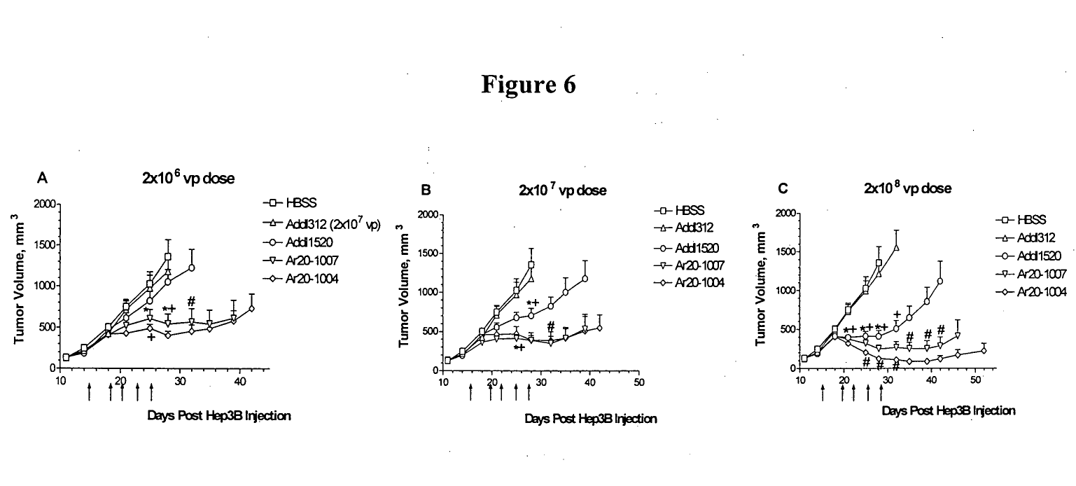

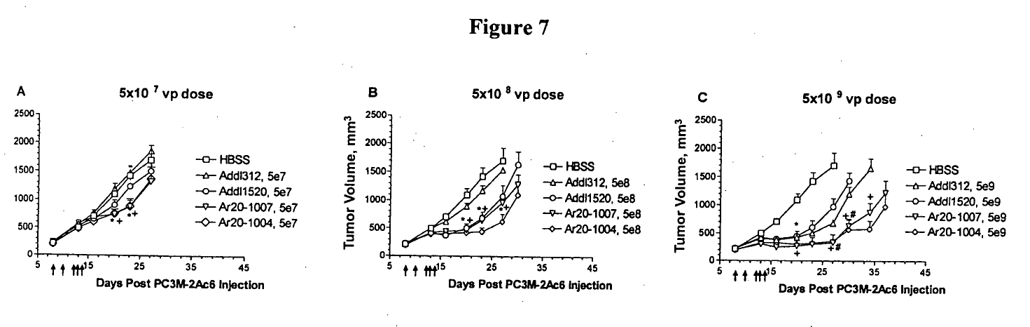

Methods. Female athymic nude (nu/nu) mice (Hep3B and PC3M.2Ac6 models) or male CB17/lcr-SCID (LnCaP model in matrigel) mice were injected subcutaneously with tumor cells when they were 6-8 weeks of age. When the tumor volumes reached 50-250 mm³ [calculated as volume=(W²xL)π/6; W, width; L, length, in cubic millimeters, animals were distributed into groups to yield similar group average tumor volumes and intratumoral injections were initiated. The viral vector dose range selected for the individual tumor models was based on the results of in vitro cytotoxicity assays. Mice were injected with viruses five times on an every other day schedule. A sham-treated group was injected with HBSS, the diluent used to prepare viral vectors. Tumors were measured twice weekly for the duration of the study. Details of particular experiments are included in description of the figures (FIGS. 6, 7, 8). Tumor volumes were calculated.

Tumor volumes (FIGS. 6, 7, 8) were compared using the SigmaStat software. The tumor volume analysis performed was repeat measures, one-way analysis of variance (RM-OW-ANOVA). The Tukey test for all pairwise comparisons was performed when the groups failed the test for normality. Dunnett's method was used to compare several treatments to a control treatment such as HBSS or the Addl312 viral vector. Group average tumor volume was recorded until more than one mouse in the group was sacrificed due to tumor growth greater than 2000 mm³. Comparisons of tumor-free mice (Table M) were performed by Fisher's exact test using SigmaStat. P values less than 0.05 were considered significant.

TABLE M

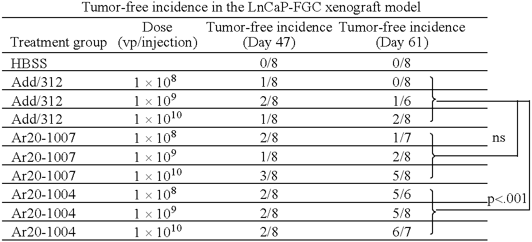

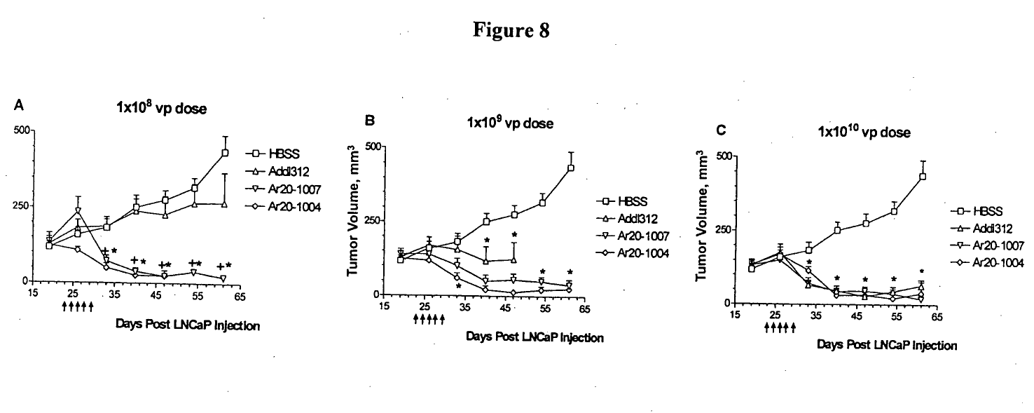

Mice were treated as described in FIG. 8. Mice were examined by palpitation and determined to be tumor-free at the initial tumor injection site. Mice were examined on Study day 47 or 61, the final day of the study. Statistical analysis by Fisher's exact test was performed on pooled groups treated with the same viral vector at different dose levels.

Results and conclusions. These studies (FIGS. 6, 7, 8, Table M) demonstrated significant anti-tumor efficacy of two Ar20 backbone oncolytic adenoviral vectors against three different subcutaneous human tumor xenografts. Both Ar20-1007 (expresses human GM-CSF) and Ar20-1004 (expresses mouse GM-CSF) were superior to Addl1520, an in-class replication-competent adenoviral vector that has been tested in phase I, II, and III human clinical trials (Ries and Korn 2002, Nemunaitis, et al 2000, Heise and Kim 2000). Similarly, Ar20-1007 and Ar20-1004 were superior to Add/312, a replication-defective adenovirus, indicating that viral replication is necessary for efficacy. The LnCaP-FGC tumor study (FIG. 8, Table M) revealed that the Ar20-1004 viral vector that expresses murine GM-CSF induced a significantly higher number of tumor-free mice at day 61 compared to Addl312. Ar20-1007 also had a trend towards greater numbers of tumor free mice, but the differences were not significant. This result demonstrated the advantage of local expression of the biologically active species relevant GM-CSF even in immunodeficient SCID mice.

In summary, Ar20-1007 and Ar20-1004 have been designed as oncolytic adenoviruses that carry most of the E3 region in which the expression of the essential E1a gene is controlled by the tumor selective E2F-1 promoter. The vector carries the packaging signal in the native location and carries a polyadenylation signal upstream of the E2F-1 promoter to inhibit transcriptional read-through from the LITR. The vector was further designed to be armed with the ability to express GM-CSF under control of the E3 promoter that is transactivated by E1A.

Increased intracellular E2F-1 levels in Rb-pathway disregulated cells have been confirmed as the target of Ar20-1007 and Ar20-1004. As a result, E1A is selectively produced in Rb-pathway disregulated cells and the E3 promoter driving GM-CSF expression is selectively activated in tumor cells as well. Human tumor cells are efficiently transduced and Ar20-1007 tumor selectivity, as measured by in vitro cytotoxicity assays, is superior to the in-class competitor Addl1520. Biologically active GM-CSF production is induced in a dose related fashion at levels known to stimulate anti-tumor protective immunity in the tumor vaccine setting (Dranoff et al., Vaccination with irradiated tumor cells engineered to secrete murine GM-CSF stimulates potent, specific and long-lasting anti-tumor immunity. Proc National Acad Sci 90:3539-3543, 1993; Simons J W, Jaffee E M, Weber C E, et. al. (1997) Bioactivity of autologous irradiated renal cell carcinoma vaccines generated by ex vivo granulocyte-macrophage colony-stimulating factor gene transfer. Cancer Res. 57:1537-1546).

Ar20-1007 and Ar20-1004 are potent antitumor agents in experimental human xenograft models. Due to the species-specific activity of GM-CSF, vectors carrying human or mouse GM-CSF were created. Ar20-1004, expressing mouse GM-CSF, demonstrated a significant enhancement to tumor-free survival in a xenograft model. Thus, even in a T cell deficient animal, the virus carrying species-matched mouse GM-CSF cDNA (i.e., Ar20-1004) showed evidence of increased efficacy relative to a virus carrying the human GM-CSF cDNA (i.e., Ar20-1007). These results may be due to the stimulation of innate immunological, inflammatory and anti-angiogenic responses (Dong et al., 1998) by mouse GM-CSF. Ar20-1007, carrying the human GM-CSF cDNA, is expected to similarly enhance the innate immune system in human cancer patients.

Strong evidence in vivo of vector spread through tumors was demonstrated in the PC3M.2Ac6 model. Two days following a single intratumoral administration of Ar20-1007, only a few percent of tumor cells contained adenoviral hexon protein. After 6 days, this number had risen to greater than 50%.

Following intratumoral administration of Ar20-1004 to mice at 1.54×10¹⁰ VP/injection, the highest dose tested, mouse GM-CSF was initially found in both the serum and the tumor at high levels. However, with time, the serum level of mouse GM-CSF declined by 4 logs, faster than the decline of tumor mouse GM-CSF levels. As with mouse GM-CSF tumor levels, human GM-CSF in the tumors was detectable at high levels throughout the course of the experiment. Serum levels of human GM-CSF initially reached a similar level as mouse GM-CSF, but then remained higher than mouse GM-CSF levels throughout the course of the study and only declined by about one log. Clinically, human GM-CSF pharmacokinetics are expected to resemble the pattern seen with mouse GM-CSF in mouse models. Thus, high levels of GM-CSF should be maintained for a considerable period of time at the site of action in the injected tumor mass, resulting in a continuous stimulation of the immune system and the presentation of tumor antigens released following local adenoviral mediated oncolysis.

The dose response studies showed that GM-CSF exhibited different kinetics depending on the viral dose administered. The lower doses (1.54×10⁶ and 1.54×10⁸ VP/injection) tended to have a flatter course, resulting in a more even exposure to the cytokine. In fact, at later time points, the three doses nearly merged in both the serum and tumor levels of GM-CSF detected.

The rough estimates of C_{MAX}, 20 pg/ml to 31.6 ng/ml in serum, derived from these studies overlaps the 5 ng/ml C_{MAX} observed following intravenous administration to healthy males of the clinical dose of 250 μg/m² intravenous Sargramostim (yeast produced recombinant human GM-CSF, Armitage, 1998). The total exposure to GM-CSF seen in the studies presented here is significantly higher at the middle and high doses administered than the 640 to 677 ng/mL min reported for a single bolus injection of Sargramostim. It is expected that the prolonged production of significant levels of GM-CSF at the tumor will result in robust anti-tumor immune responses.

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive.

### DESCRIPTION OF THE SEQUENCES IN THE SEQUENCE LISTING

The Sequence Listing associated with the instant disclosure is hereby incorporated by reference into the instant disclosure. The following is a description of the sequences contained in the Sequence Listing:

SEQ ID NO:1 is a 273 bp fragment containing sequences from the human E2F promoter.

SEQ ID NO:2 is a 397 bp fragment containing sequences from the human TERT promoter.

SEQ ID NO:3 is a 245 bp fragment containing sequences from the human TERT promoter.

SEQ ID NO:4 is nucleotides 1 to 2055 of Ar20-1007 including ITR, packaging signal, poly A, E2F-1 promoter, E1a gene and a portion of the E1b gene (FIG. 4).

SEQ ID NO:5 is nucleotides 28781 to 29952 of Ar20-1007 including the E3-6.7 gene, and the human GM-CSF cDNA (FIG. 4).

SEQ ID NO:6 is the amino acid sequence of human GM-CSF encoded by Ar20-1007 (FIG. 4).

SEQ ID NO:7 is nucleotides 28827 to 29656 of Ar20-1004 which includes a sequence encoding a mouse GM-CSF (FIG. 5).

SEQ ID NO:8 is the amino acid sequence of mouse GM-CSF encoded by Ar20-1004 (FIG. 5).

SEQ ID NO:9 is nucleotides 1 to 2038 of Ar20-1006 including an ITR, packaging signal, poly A, hTERT promoter, E1a gene and a portion of the E1b gene. (FIG. 9)

SEQ ID NO:10 is nucleotides 28772 to 29671 of Ar20-1006 which includes the E3-6.7 gene, human GM-CSF cDNA and a portion of the ADP gene. (FIG. 9)

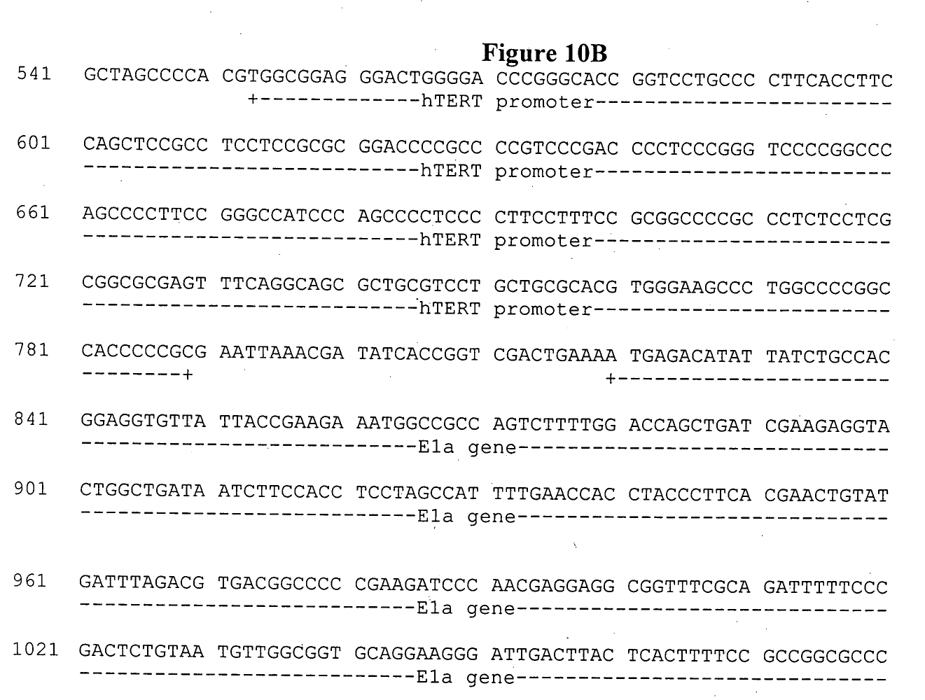

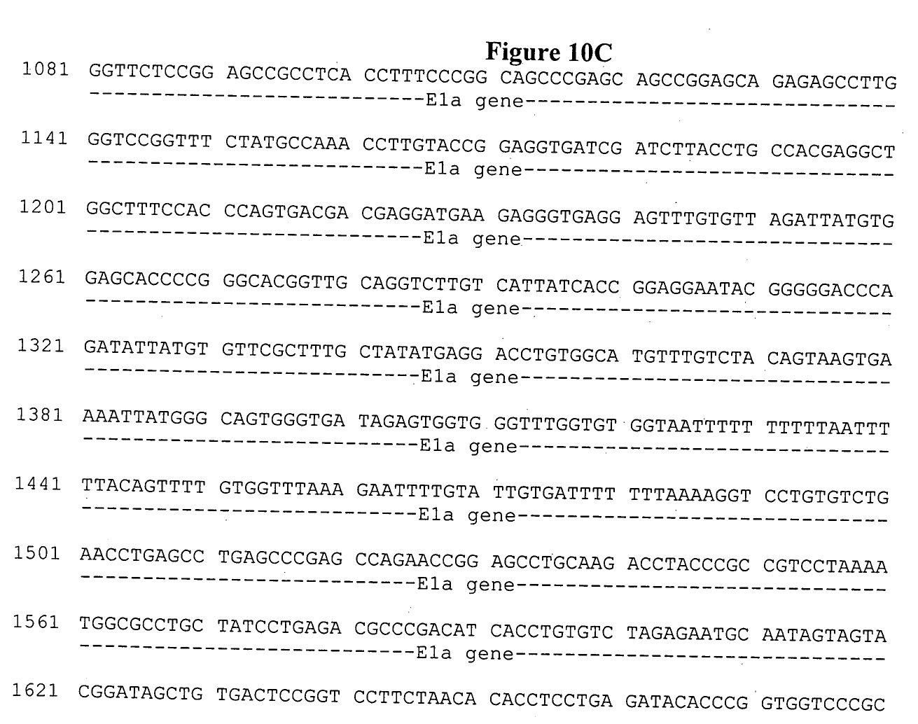

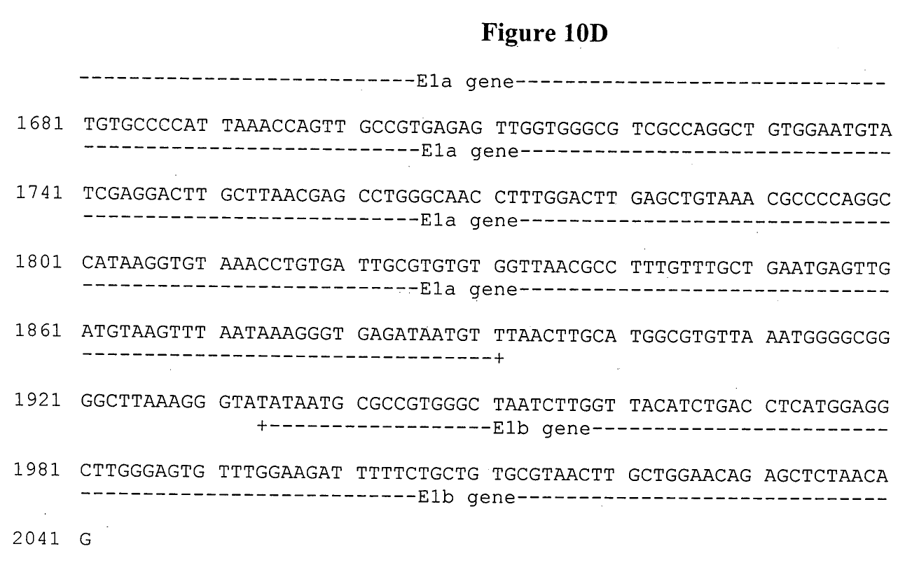

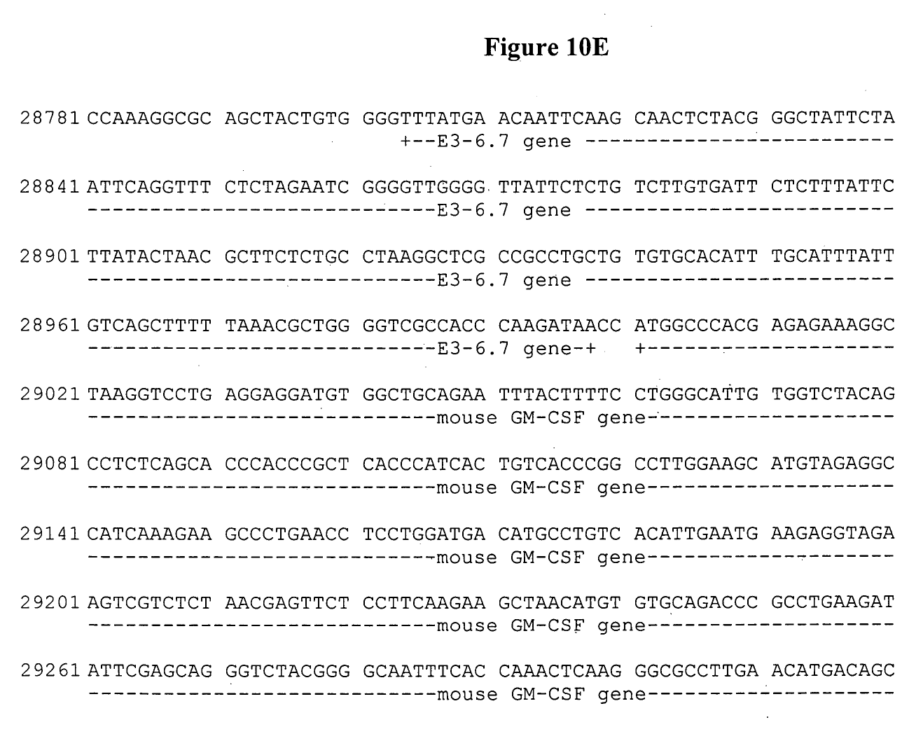

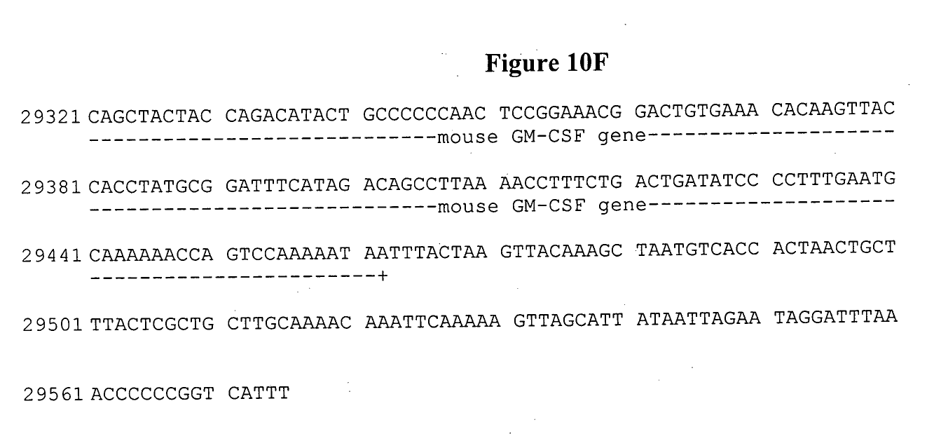

SEQ ID NO:11 is nucleotides 1 to 2041 of Ar20-1010, including an ITR, packaging signal, poly A, hTERT promoter, E1a gene and a portion of the E1b gene (FIG. 10).

SEQ ID NO:12 is nucleotides 28781 to 29575 of Ar20-1010 containing the E3-6.7 gene and the mouse GM-CSF cDNA (FIG. 10).

What is claimed is:

1. A recombinant viral vector comprising an adenoviral nucleic acid backbone, wherein said nucleic acid backbone comprises in sequential order: a left ITR, an adenoviral packaging signal, a termination signal sequence, an E2F responsive promoter operably linked to an E1a coding region, a heterologous coding sequence encoding GM-CSF and a right ITR.

2. The recombinant viral vector of claim 1, wherein the termination signal sequence is the SV40 early polyadenylation signal sequence.

3. The recombinant viral vector of claim 1, wherein the E2F responsive promoter is the human E2F-1 promoter.

4. The recombinant viral vector of claim 1, wherein the left ITR, the adenoviral packaging signal, the E1a coding region and the right ITR are derived from adenovirus serotype 5 (Ad5) or serotype 35 (Ad35).

5. The recombinant viral vector of claim 1, further comprising a mutation or deletion in the E3 region.

6. The recombinant viral vector of claim 1, wherein the E3 region has been deleted from said backbone.

7. The recombinant viral vector of claim 1, comprising SEQ ID NO:4 and SEQ ID NO:5.

8. The recombinant viral vector of claim 1, comprising SEQ ID NO:4 and SEQ ID NO:7.

9. The recombinant viral vector of claim 1, further comprising a mutation or deletion in the E1b gene.

10. The recombinant viral vector of claim 9, wherein said mutation or deletion results in the loss of the active 19 kD protein expressed by the wild-type E1b gene.

11. The recombinant viral vector of claim 1, wherein said heterologous coding sequence encoding GM-CSF is inserted in the E3 region.

12. The recombinant viral vector of claim 1, wherein said heterologous coding sequence encoding GM-CSF is inserted in place of the 19 kD E3 gene.

13. The recombinant viral vector of claim 1, wherein said heterologous coding sequence encoding GM-CSF is inserted in place of the 14.7 kD E3 gene.

14. The recombinant viral vector of claim 1, wherein said recombinant viral vector is capable of selectively replicating in and lysing Rb-pathway defective cells.

15. The recombinant viral vector of claim 14, wherein tumor-selectivity is at least about 3-fold as measured by E1A RNA levels in infected tumor vs. non-tumor cells.

16. The recombinant viral vector of claim 1, wherein said adenoviral nucleic acid backbone is an Ad5 nucleic acid backbone.

17. An adenoviral vector particle comprising the viral vector of claim 1.

18. A method of selectively killing a neoplastic cell in a cell population which comprises contacting an effective amount of the adenoviral vector particle of claim 17 with said cell population under conditions where the recombinant viral vector transduces the cells of said cell population.

19. The method of claim 18, wherein the neoplastic cell has a defect in the Rb-pathway.

20. A pharmaceutical composition comprising the adenoviral vector particle of claim 17 and a pharmaceutically acceptable carrier.

21. A method of treating a host organism having a neoplastic condition, comprising administering a therapeutically effective amount of the composition of claim 20 to said host organism.

22. The method of treatment of claim 21, wherein the host organism is a human.

23. The method of treatment of claim 21, wherein the neoplastic condition is bladder, head and neck, lung, breast, prostate, or colon cancer.

24. The vector of claim 1, wherein said backbone comprises an E3 coding region.

25. The vector of claim 24, wherein said E3 coding region is selected from the group consisting of E3-6.7, KDa, gp19KDa, 11.6 KDa (ADP), 10.4 KDa (RIDα), 14.5 KDa (RIDβ), and E3-14.7 Kda.

26. The method of treatment of claim 21, wherein administration is by intratumoral injection of a therapeutically effective dosage of the composition of claim 20.
